(19) United States
(12) Patent Application Publication
(10) Pub. No.: US 2018/0116952 A1
(43) Pub. Date: May 3, 2018

(54) ORAL PHARMACEUTICAL COMPOSITION OF METHYLERGONOVINE AND METHODS OF USE THEREOF

(71) Applicant: Lupin Atlantis Holdings SA, Zug (CH)

(72) Inventors: James Allen Garegnani, Hopewell, NJ (US); Makarand Krishnakumar Avachat, Pune (IN); Sajeev Chandran, Pune (IN); Ashish Ashokrao Deshmukh, Pune (IN); Ganesh Bhaskarrao Shete, Pune (IN)

(73) Assignee: LUPIN ATLANTIS HOLDINGS SA, Zug (CH)

(21) Appl. No.: 15/821,644

(22) Filed: Nov. 22, 2017

Related U.S. Application Data
(63) Continuation-in-part of application No. 15/160,831, filed on May 20, 2016.
(60) Provisional application No. 62/164,052, filed on May 20, 2015.

(51) Int. Cl. A61K 9/00; A61K 9/16; A61K 9/28; A61K 9/48; A61K 31/48; A61P 25/06; A61P 7/04

(52) U.S. Cl. CPC A61K 9/0053; A61K 9/1623; A61K 9/1676; A61K 9/282; A61K 9/2846; A61P 7/04; A61K 9/4808; A61K 9/0002; A61K 31/48; A61P 25/06; A61K 9/2866

(57) ABSTRACT

A solid pharmaceutical oral composition for once or twice daily administration is provided. The composition includes pulsed release pellets. Each pellet includes at least a first drug layer comprising methylergonovine or a pharmaceutically acceptable salt thereof, and a second drug layer comprising methylergonovine or a pharmaceutically acceptable salt thereof. The solid pharmaceutical oral composition includes from about 0.3 mg to about 0.8 mg in total of methylergonovine or a pharmaceutically acceptable salt thereof. The composition is used for treating a subject having a methylergonovine responsive condition such as migraine, refractory migraine, uterine atony, uterine haemorrhage, subinvolution of the uterus, and uterine haemorrhage in the second stage of labor.

### CROSS-REFERENCE TO RELATED APPLICATIONS

This application is a continuation-in-part (“CIP”) application of U.S. patent application Ser. No. 15/160,831, filed on May 20, 2016, which claims the benefit of and priority to U.S. Provisional Application No. 62/164,052, filed on May 20, 2015. All applications above and any references or products mentioned below are expressly incorporated by reference herein in their entireties.

### BACKGROUND OF THE INVENTION

### (a) Field of the Invention

The present invention is directed to an oral modified release pharmaceutical composition of methylergonovine suitable for once or twice daily administration. The composition comprises at least about 0.6 mg dose of methylergonovine and is suitable for once daily administration. Alternatively, the composition comprises at least about 0.3 mg dose of methylergonovine and is suitable for twice daily administration. The invention is further directed to use of said composition for the treatment of methylergonovine responsive conditions such as migraine, refractory migraine, uterine atony, uterine haemorrhage, subinvolution of the uterus, and uterine hemorrhage in the second stage of labor. Particularly, the present invention provides a method of using said composition for treating migraine, refractory migraine or management of uterine atony, hemorrhage and subinvolution of the uterus.

### (b) Description of the Related Art

Methylergonovine is a semisynthetic analogue of ergonovine, a psychedelic alkaloid found in ergot, and many species of morning glory. The marketed products contain the maleate salt of methylergonovine. Methylergonovine maleate is an active metabolite of methysergide, a drug well known for its value in migraine and cluster headache prevention but which is currently not available. Methylergonovine is a smooth muscle constrictor that mostly is used as a uterine contraction drug. It is most commonly used to prevent or control excessive bleeding following childbirth and spontaneous or elective abortion, and also to aid in expulsion of retained products of conception after a missed abortion (miscarriage in which all or part of the foetus remains in the uterus) and to help deliver the placenta after childbirth.

Methylergonovine is available as injection (intramuscular or intravenous) or as liquid and tablets to be taken orally. The molecular formula of methylergonovine maleate is C₂₀H₂₅N₃O₂.C₄H₄O₄, and its structural formula is:

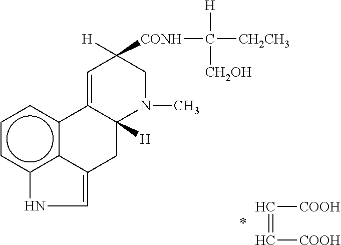

Currently, tablet and injection dosage forms of methylergonovine maleate are available in the United States under as the brand Methergine® from Novartis. Methergine® tablets are available in the 0.2 mg strength and Methergine® injection is available in 0.1 mg and 0.2 mg strengths. For effective treatment of uterine atony, haemorrhage and subinvolution of the uterus, and control of uterine haemorrhage and as per the administration recommendation, the tablets are administered 3-4 times daily and injection is administered at intervals of 2-4 hours.

Due to its vasoconstriction properties, Methergine® is also recommended for several situations as related to some headache patients. Particularly, it is recommended for treating vascular headaches, such as migraine (including menstrual migraine) or cluster.

Migraine headaches are a potentially chronic, progressive and pervasive disease that erodes the sufferer's daily quality of life as well as substantially affecting patients' families, workplaces and society. Historically, migraine has been characterized as episodic attacks separated by normal, symptom-free periods. Findings from migraine sufferers surveyed in a national poll revealed that migraineurs do not view their migraines as isolated events. These sufferers consider their migraines part of a cycle of suffering, treating their current attack and worrying about when the next attack will strike. (J. L. Brandes, Headache: The Journal of Head and Face Pain; Vol. 48, p. 430-441, March 2008).

Effect of methylergonovine in treating refractory migraine has been also studied. Graff-Radford and Bittar reported on 60 consecutive patients with what the authors called drug-induced refractory headache; the authors suggested that methylergonovine was effective in 73% of the patients (S B Graff-Radford, G T Bittar; The use of methylergonovine in the initial control of drug-induced refractory headache; Headache, 1993; 33 (7): 390-393).

The role of methylergonovine in treating refractory migraine: has been recently evaluated by Saper and Evans (Joel R. Saper, Randolph W. Evans; Oral methylergonovine maleate for refractory migraine and cluster headache prevention; Headache, 2013; 53(2): 378-381).

There is no standard definition of refractory migraine available and the condition is judged on the basis of symptoms. Refractory migraine is understood to describe a variety of clinical symptoms associated with persistent headache that is difficult to treat or fails to respond to standard headache treatments.

The currently available oral dosage forms of methylergonovine require multiple daily administration for treatment. Particularly, tablets containing 0.2 mg to 0.4 mg of methylergonovine are required to be administered three times daily, and in some cases four times daily administration is suggested to narrow the interval between dosing. In case of chronic migraine treatment, patients are given a first dose of methylergonovine maleate followed by further multiple doses until the patient experiences relief from pain. The need of such frequent administration of currently available products, however, may result in less preferred treatment choices for many patients, mainly due to compliance issues and especially in case of geriatric patients. Similarly in case of administration of Methergine® following delivery of placenta, for routine management of uterine atony, hemorrhage and subinvolution of the uterus, it would be beneficial to reduce the multiple daily administration of four times day to twice daily or once daily to increase patient compliance and ease of administration.

Further, there are adverse effects associated with currently available products and their dosing regimen. The most common adverse effect is hypertension associated in several cases with seizure and/or headache. Hypotension has also been reported. Abdominal pain (caused by uterine contractions), nausea and vomiting have occurred occasionally.

There remains a need for an oral composition containing a unique dose and an improved formulation to deliver methylergonovine. The composition advantageously needs to be administered once or twice per day and provide round-the-clock management of methylergonovine responsive conditions, including migraine and refractory migraine. Such composition should also provide equal or more therapeutic benefits than those achieved by multiple administrations of currently known 0.2 mg methylergonovine dosage forms.

### SUMMARY OF THE INVENTION

The present invention provides an oral modified release pharmaceutical composition of methylergonovine suitable for once or twice daily administration.

In one aspect, the invention provides an oral modified release pharmaceutical composition suitable for once daily administration comprising at least about 0.6 mg of methyl ergonovine or a pharmaceutically acceptable salt thereof.

In another aspect, the invention provides an oral modified release pharmaceutical composition suitable for once daily administration comprising about 0.5 mg, about 0.6 mg, about 0.8 mg, about 1 mg, about 1.2 mg, about 1.4 mg, about 1.6 mg, about 1.8 mg or about 2 mg of methylergonovine or a pharmaceutically acceptable salt thereof, as well as fractional values between these stated values.

In another aspect, the invention provides an oral modified release pharmaceutical composition suitable for twice daily administration comprising about 0.3 mg to about 0.6 mg of methyl ergonovine or a pharmaceutically acceptable salt thereof. In the preferred embodiment, the oral modified release pharmaceutical composition suitable for twice daily administration comprising about 0.4 mg, about 0.45 mg, of methylergonovine or a pharmaceutically acceptable salt thereof, as well as fractional values between these stated values.

In another aspect, the invention provides an oral modified release pharmaceutical composition suitable for once daily administration comprising at least about 0.6 mg methylergonovine or a pharmaceutically acceptable salt thereof, wherein methylergonovine or a pharmaceutically acceptable salt thereof in the composition is released from the composition over a period ranging from about 0 hour to up to about 24 hours.

In another aspect, the invention provides an oral modified release pharmaceutical composition suitable for once daily administration comprising at least about 0.6 mg methylergonovine or a pharmaceutically acceptable salt thereof, wherein methylergonovine or a pharmaceutically acceptable salt thereof in the composition is released in an extended release manner, with or without an initial load dose.

In another aspect, the invention provides an oral modified release pharmaceutical composition comprising:
(a) at least one extended release portion comprising methylergonovine or a pharmaceutically acceptable salt thereof, and
(b) optionally, at least one immediate release portion comprising methylergonovine or a pharmaceutically acceptable salt thereof.

In another aspect, the invention provides an oral modified release pharmaceutical composition suitable for once daily administration comprising:
(a) a core comprising at least about 0.4 mg to about 1.6 mg methylergonovine or a pharmaceutically acceptable salt thereof exhibiting extended release, and
(b) at least one layer surrounding the core comprising at least about 0.2 mg to about 0.6 mg methylergonovine or a pharmaceutically acceptable salt thereof exhibiting immediate release.

In another aspect, the invention provides an oral modified release pharmaceutical composition suitable for once daily administration comprising:
(a) at least one immediate release portion comprising at least about 0.2 mg to about 0.6 mg methylergonovine or a pharmaceutically acceptable salt thereof, and
(b) at least one extended release portion comprising about 0.4 mg to about 1.6 mg methylergonovine or a pharmaceutically acceptable salt thereof.

In another aspect, the immediate release portion of the composition exhibits complete release of methylergonovine or a pharmaceutically acceptable salt thereof within about 1 hour after oral administration.

In another aspect, the extended release portion of the composition exhibits release of methylergonovine or a pharmaceutically acceptable salt thereof over a period of up to about 24 hours after oral administration.

In another aspect, the release of methylergonovine or pharmaceutically acceptable salt thereof from the extended release portion of the composition starts about 2 hours, about 4 hours, about 6 hours or about 8 hours after oral administration.

In another aspect, the invention provides an oral modified release pharmaceutical composition suitable for once daily administration comprising:
(a) a core comprising at least about 0.4 mg to about 1.6 mg of methylergonovine or a pharmaceutically acceptable salt thereof exhibiting extended release, and
(b) at least one layer surrounding the core comprising at least about 0.2 mg to about 0.6 mg of methylergonovine or a pharmaceutically acceptable salt thereof exhibiting immediate release.

In another aspect, the invention provides a solid oral unit dosage form suitable for once daily administration comprising:
(a) at least one layer comprising least about 0.2 mg to about 0.6 mg methylergonovine or a pharmaceutically acceptable salt thereof exhibiting immediate release, and
(b) at least one layer comprising about 0.4 mg to about 1.6 mg methylergonovine or a pharmaceutically acceptable salt thereof exhibiting extended release.
In another aspect, the invention provides a method of treating a condition selected from migraine, refractory migraine, uterine atony, uterine haemorrhage, subinvolution of the uterus, or uterine haemorrhage in the second stage of labor. The method comprises once daily oral administration of a modified release pharmaceutical composition comprising at least about 0.6 mg dose of methylergonovine or pharmaceutically acceptable salt thereof.

In another aspect, the invention provides a method of treating migraine or refractory migraine. The method comprises once daily oral administration of a modified release pharmaceutical composition comprising at least about 0.6 mg dose of methylergonovine or pharmaceutically acceptable salt thereof.

In another aspect, the invention provides a method for the treatment of refractory migraine comprising orally administering to a patient in need thereof the modified release pharmaceutical composition as described herein.

In another aspect, the invention provides a method for the management of uterine atony, hemorrhage and subinvolution of the uterus comprising orally administering to a patient in need thereof the modified release pharmaceutical composition as described herein.

In another aspect, the present disclosure provides an oral pharmaceutical composition suitable for once or twice daily administration, the method of making and the method of using such composition. The oral pharmaceutical compositions suitable for once or twice daily administration may have any of the structures as described herein.

In accordance with some embodiments, a solid pharmaceutical oral composition is configured for twice daily administration, and comprises from about 0.3 mg to about 0.6 mg (e.g., 0.4 mg or 0.45 mg) in total of methylergonovine, a pharmaceutically acceptable salt thereof (e.g., methylergonovine maleate), or a combination thereof. Such a solid pharmaceutical oral composition comprises an extended release matrix comprising methylergonovine or a pharmaceutically acceptable salt thereof. The extended release matrix may comprise a hydrophilic release rate controlling compound, a hydrophobic release rate controlling compound, or a combination thereof. The extended release matrix comprises an intragranular portion comprising methylergonovine or a pharmaceutically acceptable salt thereof, at least one excipient, and optionally at least one release rate controlling compound; and an extragranular portion comprising at least one release rate controlling compound. In an embodiment, the extended release matrix comprises methylergonovine or a pharmaceutically acceptable salt thereof and at least one release rate controlling compound. The release rate controlling compound can be present intragranularly or extragranularly or both. In some embodiments, such a solid pharmaceutical oral composition further comprises an immediate release layer comprising methylergonovine or a pharmaceutically acceptable salt thereof (e.g., about 0.1 mg). The immediate release layer is at least partially disposed on the extended release matrix. The solid pharmaceutical oral composition can have a bilayer structure.

In some embodiments, such a solid pharmaceutical oral composition further comprises an immediate release overcoat comprising methylergonovine or a pharmaceutically acceptable salt thereof (e.g., about 0.1 mg). The immediate release overcoat is disposed on and covers the extended release matrix.

In some embodiments, such a solid pharmaceutical oral composition further comprises an extended release coat, and an intermediate release overcoat. The extended release coat comprises a hydrophilic release rate controlling compound, a hydrophobic release rate controlling compound, or combination thereof. The extended release coat is disposed on and covers the extended release matrix. The immediate release overcoat comprises methylergonovine or a pharmaceutically acceptable salt thereof (e.g., about 0.1 mg), and is disposed on and covers the extended release coat

Such a solid pharmaceutical oral composition may be in a form of a tablet; a capsule; granules; pellets; powder; or granules, pellets and/or powder filled in a capsule. In some embodiments, such a dosage form is a tablet.

A method of making such a solid pharmaceutical oral composition comprises one or more steps such as preparing an extended release matrix, forming an immediate release layer or overcoat, and forming an extended release coat as described above.

In accordance with some embodiments, the present disclosure also provides a solid pharmaceutical oral composition configured for once daily administration, comprising from about 0.5 mg to about 0.8 mg (e.g., 0.5 mg, 0.6 mg or 0.7 mg) in total of methylergonovine or a pharmaceutically acceptable salt thereof. Such a dosage form may also have any of the structural configurations as described above.

In accordance with some embodiments, a solid pharmaceutical oral composition is configured for once or twice daily administration, and comprises pulsed release pellets. Each pellet comprising a core comprising methylergonovine or a pharmaceutically acceptable salt thereof or at least a first drug layer comprising methylergonovine or a pharmaceutically acceptable salt thereof, and a second drug layer comprising methylergonovine or a pharmaceutically acceptable salt thereof. The solid pharmaceutical oral composition comprises from about 0.3 mg to about 0.8 mg in total of methylergonovine or a pharmaceutically acceptable salt thereof. Each pellet may further comprise an inert core, e.g. a sugar core or microcrystalline cellulose core. The first drug layer is disposed on and covers the inert core.

In some embodiments, each pellet further comprises an extended release coating comprising at least a first release rate controlling polymer disposed between the first drug layer and the second drug layer, and a delayed release coating comprising at least a second release rate controlling polymer disposed on the second drug layer. The first release rate controlling polymer in the extended release coating may comprise hydroxypropyl methylcellulose and/or ethyl cellulose in some embodiments. The second release rate controlling polymer in the delayed release coating may comprise at least one copolymer of methacrylic acid and methyl methacrylate in some embodiments. The first and the second rate controlling polymers may be the same or different. Each of the extended release coating and the delayed release coating may comprise a hydrophilic release rate controlling compound, a hydrophobic release rate controlling compound, or a combination thereof.

In some embodiments, each pellet further comprises an immediate release layer comprising methylergonovine or a pharmaceutically acceptable salt thereof disposed on and covering the delayed release coating. The solid pharmaceutical oral composition may be a tablet including compressed pulsed release pellets, or a capsule filled with the pulsed release pellets. The capsule may optionally comprise an exterior immediate release coating comprising methylergonovine or a pharmaceutically acceptable salt thereof.

Such a solid pharmaceutical oral composition may comprise from about 0.6 mg to about 0.7 mg in total of methylergonovine or a pharmaceutically acceptable salt thereof, and is configured for once daily administration. In some embodiments, the solid pharmaceutical oral composition comprises from about 0.4 mg to about 0.45 mg in total of methylergonovine or a pharmaceutically acceptable salt thereof, and is configured for twice daily administration.

The present disclosure also provides a method of making the solid pharmaceutical oral composition, comprising preparing the pulsed release pellets. The method may further comprise compressing the pulsed release pellets into a tablet, or filling the pulsed release pellets into a capsule. Optionally, the method may further comprise coating the capsule with an exterior immediate release coating comprising methylergonovine or a pharmaceutically acceptable salt thereof.

In accordance with some embodiments, the present disclosure provides a solid pharmaceutical oral composition configured for once or twice daily administration, and comprising an immediate release core, and a polymer coating. The immediate release core comprises methylergonovine or a pharmaceutically acceptable salt thereof. The polymer coating is disposed on the immediate release core. The solid pharmaceutical oral composition comprises from about 0.3 mg to about 0.8 mg in total of methylergonovine or a pharmaceutically acceptable salt thereof. The solid pharmaceutical oral composition further comprises an immediate release layer comprising methylergonovine or a pharmaceutically acceptable salt thereof, which is disposed on the polymer coating. The polymer coating comprises at least one release rate controlling polymer. The percentage of coating on the core tablet (immediate release core or extended release core) depends on the required release of the drug.

In some embodiments, the polymer coating is an extended release coating comprises a first release rate controlling polymer, for example, hydroxypropyl methylcellulose and/or ethyl cellulose.

In some embodiments, the polymer coating is a semipermeable osmotic coating comprising a second release rate controlling polymer, for example, cellulose acetate phthalate. The polymer coating may optionally define orifices for allowing release of methylergonovine or a pharmaceutically acceptable salt thereof from the immediate release core. The orifices can be laser drilled or can be made by osmotic agent(s), which are soluble and create orifices in the polymer coating.

Such a solid pharmaceutical oral composition may be a tablet; a capsule; granules; pellets; powder; or granules, pellets, powder or a combination thereof filled in a capsule. In some embodiments, such a solid pharmaceutical oral composition is in a form of a tablet. In some embodiments, the solid pharmaceutical oral composition comprises from about 0.6 mg to about 0.7 mg in total of methylergonovine or a pharmaceutically acceptable salt thereof, and is configured for once daily administration. In some other embodiments, the solid pharmaceutical oral composition comprises from about 0.4 mg to about 0.45 mg in total of methylergonovine or a pharmaceutically acceptable salt thereof, and is configured for twice daily administration.

The present disclosure also provide a method of making the solid pharmaceutical oral composition, comprising one or more steps including preparing the immediate release core, coating the immediate release core with the polymer coating, and forming an immediate release coating as described herein.

The twice daily formulations or dosage forms described herein are configured to provide a dissolution profile of a release of methylergonovine or a pharmaceutically acceptable salt thereof

within 0.5 hours being between about 10% and about 35%,

within 3 hours being between about 30% and about 60%,

within 6 hours being between about 40% and about 85%, and

within 10 hours being not less than 70%,

as measured by a dissolution method employing a USP Type-II dissolution apparatus equipped with a paddle, a rotation speed of 75 rpm and 900 mL of tartaric acid (1 in 200 w/w) as dissolution medium.

The once daily formulations or dosage forms described herein are configured to provide a dissolution profile of a release of methylergonovine or a pharmaceutically acceptable salt thereof

within 0.5 hours being between about 5% and about 25%,

within 2 hours being between about 15% and about 45%,

within 6 hours being between about 25% and about 65%,

within 10 hours being between about 35% and about 85%, and

within 16 hours being not less than 75%,

as measured by a dissolution method employing a USP Type-II dissolution apparatus equipped with a paddle, a rotation speed of 75 rpm and 900 mL of tartaric acid (1 in 200 w/w) as dissolution medium.

The formulations and dosage forms described herein can be used for treating a subject having a methylergonovine responsive condition comprising a step of administering to the subject in need thereof a therapeutically effective amount of a one or more of the formulations and dosage forms discussed herein. The methylergonovine responsive condition is selected from migraine, refractory migraine, uterine atony, uterine haemorrhage, subinvolution of the uterus, or uterine haemorrhage in the second stage of labor. Depending on the dosage amount, the dosage forms can be administrated once or twice daily orally. The subject is an animal, mammal or human.

Still other aspects and advantages of the invention will be apparent from the following detailed description of the invention.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a cross-sectional view illustrating an exemplary monolithic matrix tablet comprising an oral pharmaceutical composition in accordance with some embodiments.

FIG. 2 is a cross-sectional view illustrating an exemplary bilayer tablet in accordance with some embodiments.

FIG. 3 is a cross-sectional view illustrating an exemplary tablet comprising an ER matrix and an IR over coat in accordance with some embodiments.

FIG. 4 is a cross-sectional view illustrating an exemplary tablet comprising an ER matrix, an ER coat, and an IR over coat in accordance with some embodiments.

FIG. 5 is a cross-sectional view illustrating an exemplary capsule filled with pulsed release pellets in accordance with some embodiments.

FIG. 6 is a cross-sectional view illustrating an exemplary tablet into which pulsed release pellets are compressed in accordance with some embodiments.

FIG. 7 is a cross-sectional view illustrating an exemplary capsule filled with pulsed release pellets and comprising an IR coat in accordance with some embodiments.

FIG. 8 is a cross-sectional view illustrating an exemplary tablet comprising an IR core or reservoir, an ER coat, and an IR overcoat in accordance with some embodiments.

FIG. 9 is a cross-sectional view illustrating an exemplary osmotic tablet having an IR overcoat in accordance with some embodiments.

FIG. 10 shows the dissolution profiles of three examples of the exemplary monolithic matrix tablet (as illustrated in FIG. 1) comprising 0.4 mg of methylergonovine maleate.

FIG. 11 shows the dissolution profiles of three examples of the exemplary monolithic matrix tablet (as illustrated in FIG. 1) comprising 0.7 mg of methylergonovine maleate.

### DETAILED DESCRIPTION OF THE INVENTION

The following disclosure provides many different embodiments, or examples, for implementing different features of the invention. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of a tablet in addition to the orientation depicted in the figures. The tablet may be otherwise oriented (rotated 90 degrees or at other orientations) and the spatially relative descriptors used herein may likewise be interpreted accordingly.

In the present disclosure the singular forms “a,” “an,” and “the” include the plural reference, and reference to a particular numerical value includes at least that particular value, unless the context clearly indicates otherwise. Thus, for example, a reference to “a layer” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2. to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” it is intended that any component, element, attribute, or step that is positively recited herein may be explicitly excluded in the claims, whether such components, elements, attributes, or steps are listed as alternatives or whether they are recited in isolation.

The invention provides an oral modified release pharmaceutical composition suitable for once daily administration of methylergonovine or a pharmaceutically acceptable salt thereof. Preferably, the composition contains at least about 0.6 mg of methylergonovine or a pharmaceutically acceptable salt thereof, and preferably the maleate salt thereof.

The invention addresses the need for a composition of methylergonovine which requires only once daily administration and provides relief which is comparable to that achieved by single as well as multiple administrations of currently available methylergonovine maleate 0.2 mg tablets, including Methergine®.

A once daily administration of methylergonovine is advantageous over a multiple administration dosing regimen in terms of both patient compliance and potential reduction in adverse events associated with the drug, thereby providing better treatment of the conditions for which methylergonovine chloride or methylergonovine maleate is indicated.

The inventors have devised a unique oral modified release pharmaceutical composition in order to provide for an effective once or twice daily form of methylergonovine that may provide round-the-clock desired therapeutic effects while minimizing, if not eliminating, the undesired side effects.

The oral modified release pharmaceutical composition of the invention comprises at least about 0.6 mg methylergonovine, a pharmaceutically acceptable salt thereof, or a combination thereof, and is suitable for once daily administration. Alternatively, the oral modified release pharmaceutical composition of the invention comprises at least about 0.3 mg methylergonovine, a pharmaceutically acceptable salt thereof, or a combination thereof; and is suitable for twice daily administration.

The term “pharmaceutically acceptable salt” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids (e.g. maleate, tartrate), phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like. A preferred salt is the maleate salt.

The term “modified release” as used herein in relation to the composition according to the invention means release, which is not immediate release as such and is taken to encompass controlled release, sustained release, prolonged release, timed release, retarded release, extended release and delayed release or combinations thereof with immediate release. The term “modified release dosage form” as used herein can be described as dosage forms whose drug-release characteristics of time course and/or location are chosen to accomplish therapeutic or convenience objectives not offered by conventional dosage forms such as a solution or an immediate release dosage form. Modified release solid pharmaceutical oral compositions include both delayed and extended release drug products (as per US FDA guideline for ‘SUPAC-MR: Modified Release Solid Oral Dosage Forms’).

The “immediate release” refers to the release of the active ingredient over a period of time less than 1 hour after initiation of the release.

As used herein, the term “extended release” refers to the release of the active ingredient over an extended period of time leading to relatively lower peak plasma concentrations and a prolonged bioavailability as compared to “immediate release” compositions of the same active ingredient.

The term “portion” refers to mini-tablet, tablet, pellet, bead, granule, a layer of a tablet, a coated layer, powder or any other known solid physical form prepared by standard methods known to the person skilled in the art, including but not limited to compression, granulation, spray coating, prilling, and extrusion/spheronization.

As used herein, the term “a therapeutic effective amount” refers to a dosage or amount of a solid pharmaceutical oral composition as described herein to provide desired effect in treating a disease without toxic effects or with minimal side effects within normal acceptable safety ranges. A composition is effective in treatment of methylergonovine responsive conditions such as migraine, refractory migraine, uterine atony, uterine haemorrhage, subinvolution of the uterus, and uterine hemorrhage in the second stage of labor. Particularly, such a composition is effective in treating migraine, refractory migraine or management of uterine atony, hemorrhage and subinvolution of the uterus.

In an embodiment, the oral modified release pharmaceutical composition releases methylergonovine or a pharmaceutically acceptable salt thereof from the composition over a period ranging from about 0 hour to up to about 24 hours.

In a further embodiment, a portion of methylergonovine or a pharmaceutically acceptable salt thereof in the composition is released in a slow, continuous release manner and a remaining portion provides an initial load dose of methylergonovine or a pharmaceutically acceptable salt thereof.

In a further embodiment, the complete amount of methylergonovine or a pharmaceutically acceptable salt thereof in the composition, preferably in the extended release portion, is released in a slow, continuous release, without any initial load dose.

The oral pharmaceutical composition of the invention comprises up to 2 mg and preferably at least about 0.6 mg methylergonovine or a pharmaceutically acceptable salt thereof. The dose of methylergonovine in the composition may be about 0.6 mg, about 0.8 mg, about 1 mg, about 1.2 mg, about 1.4 mg, about 1.6 mg, about 1.8 mg or about 2 mg. The total dose may be provided as a single portion or multiple portions in the composition.

In an embodiment, the total dose of at least about 0.6 mg methylergonovine or a pharmaceutically acceptable salt thereof in the composition exhibits extended release.

In another embodiment, the composition comprises at least one portion of methylergonovine or a pharmaceutically acceptable salt thereof that exhibits extended release and at least one portion of methylergonovine or a pharmaceutically acceptable salt thereof in the composition that exhibits immediate release.

In a further embodiment, the composition comprises at least about 0.4 mg to about 1.6 mg dose of methylergonovine or a pharmaceutically acceptable salt thereof that exhibits extended release and at least about 0.2 mg to about 0.6 mg methylergonovine or a pharmaceutically acceptable salt thereof that exhibits immediate release.

The dose of methylergonovine or a pharmaceutically acceptable salt thereof in the immediate release portion may be about 0.1 mg, about 0.2 mg, about 0.3 mg or about 0.4 mg.

The dose of methylergonovine or a pharmaceutically acceptable salt thereof in the extended release portion may be about 0.2 mg, about 0.3 mg, 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg or about 1.6 mg.

The oral pharmaceutical composition or modified release composition of the invention is preferably in the form of a solid dosage form. Such dosage forms include a tablet, a coated tablet, a multilayer (e.g. bilayer and trilayer) tablet, a capsule, a caplet, granules, pellets, minitablets, powder, and granules, pellets or powder filled into a capsule.

In an embodiment, the oral pharmaceutical composition of methylergonovine or a pharmaceutically acceptable salt thereof comprises:
(a) at least one immediate release portion comprising least about 0.2 mg to about 0.6 mg methylergonovine or a pharmaceutically acceptable salt thereof, and
(b) at least one extended release portion comprising about 0.4 mg to about 1.6 mg methylergonovine or a pharmaceutically acceptable salt thereof.

In another embodiment, the modified release oral pharmaceutical composition of methylergonovine or a pharmaceutically acceptable salt thereof comprises:
(a) a core comprising at least about 0.4 mg to about 1.6 mg methylergonovine or a pharmaceutically acceptable salt thereof exhibiting extended release, and
(b) at least one layer surrounding the core comprising least about 0.2 mg to about 0.6 mg methylergonovine or a pharmaceutically acceptable salt thereof exhibiting immediate release.

In another embodiment, the modified release oral pharmaceutical composition of methylergonovine or a pharmaceutically acceptable salt thereof comprises:
(a) at least one layer comprising least about 0.2 mg to about 0.6 mg methylergonovine or a pharmaceutically acceptable salt thereof exhibiting immediate release, and
(b) at least one layer comprising about 0.4 mg to about 1.6 mg methylergonovine or a pharmaceutically acceptable salt thereof exhibiting extended release.

The inventors further observed that the oral pharmaceutical composition of the invention provides release of methylergonovine over a period of up to about 24 hours. Such release profile is particularly desirable for patients who require round-the-clock therapeutic benefit, especially for patients suffering from migraine and refractive migraine.

In an embodiment, the immediate release portion, if present in the composition, exhibits complete release of methylergonovine or a pharmaceutically acceptable salt thereof within 1 hour after oral administration.

In another embodiment, the extended release portion of the composition exhibits release of methylergonovine or a pharmaceutically acceptable salt thereof over a period of up to about 24 hours after oral administration.

In a further embodiment, release of methylergonovine or pharmaceutically acceptable salt thereof from the extended release portion of the composition starts about 2 hours, about 4 hours, about 6 hours or about 8 hours after oral administration.

The invention further provides a method of treating various conditions responsive to methylergonovine, including a condition selected from migraine, refractory migraine, uterine atony, uterine haemorrhage, subinvolution of the uterus, or uterine haemorrhage in the second stage of labor.

In an embodiment, the method of treating migraine, refractory migraine, uterine atony, uterine haemorrhage, subinvolution of the uterus, or uterine haemorrhage in the second stage of labor comprises once daily oral administration of the pharmaceutical composition comprising at least about 0.6 mg dose of methylergonovine or pharmaceutically acceptable salt thereof as substantially described throughout the specification.

In a preferred embodiment, the method of treating refractory migraine comprises once daily oral administration of the pharmaceutical composition comprising at least about 0.6 mg dose of methylergonovine or pharmaceutically acceptable salt thereof as substantially described throughout the specification.

The modified release formulations of methylergonovine exhibit an IR profile, or a XR profile, or a combination of a XR profile with an IR profile. In some embodiments, the formulations may exhibit a pulsatile release profile. These specific release profiles are achieved by formulating methylergonovine with at least one of a release rate controlling compound and at least one excipient in a variety of inventive formulations.

The release rate controlling compounds of the current invention may be selected from hydrophilic rate controlling compounds (or called hydrophilic compounds) and hydrophobic rate controlling compounds (or called hydrophobic compounds). The following non-limiting examples of such compounds are provided below.

Hydrophilic compounds may include one or more of the following: hydroxypropyl cellulose, hypromellose (hydroxypropyl methyl cellulose), methyl cellulose, polyethylene oxide, acacia, acrylic acid derivatives, alginic acid (and its salts and derivatives thereof), hydroxyethyl cellulose, povidone, carrageenan, carboxymethylcellulose, tragacanth, polyvinyl alcohol, xanthan gum and combinations thereof.

Hydrophobic compounds may include one or more of the following: ethyl cellulose, cellulose acetate, cellulose acetate butyrate, waxes (e.g., carnauba wax, microcrystalline wax), hydrogenated vegetable oils, Compritol 888 ATO (glyceryl behenate), Precirol ATO 5 (glyceryl palmitostearate), PEG glyceryl esters such as Gelucire 50/1, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer, Type A) polyvinyl acetate, cellulose acetate propionate, and combinations thereof. In an embodiment, the amount of release rate controlling compounds in the modified release oral pharmaceutical composition of methylergonovine or a pharmaceutically acceptable salt thereof ranges from about 5% to about 95%, preferably from about 10% to about 85%, and more preferably from about 15% to about 75% by weight of the composition.

Compounds that can be used as release rate controlling coatings or polymer coatings may include one or more of the following: cellulose sters, cellulose acetate, cellulose acetate butyrate, ethyl cellulose, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer Type, A), ethyl acrylate methyl methacrylate copolymer, polyvinyl acetate, shellac, zein and combinations thereof.

The application of a release rate controlling compound coating is achieved using standard coating techniques such as spraying, dipping, casting, coating solvent evaporation, molding or compression coating.

The release rate controlling compounds described above may be used to prepare a variety of modified release systems, including:

(A) Matrix Systems—wherein an active pharmaceutical ingredient (methylergonovine), at least one release rate controlling compound, and at least one pharmaceutically acceptable excipient are homogeneously intermixed to form a matrix. Hydrophilic and hydrophobic compounds listed above may be used to prepare these methylergonovine-containing matrices. These matrices may be presented in the form of matrix tablets, matrix multiparticulates, or in the form of a layer coated onto a substrate.

Matrix tablets may be in the form of multiple layer tablets (e.g., bilayer or tri-layer tablets), tablet within a tablet, encapsulated mini-tablets or a tablet of compressed modified release particles. These matrix systems may be coated with release rate controlling compounds to add additional release rate controlling characteristics or a delayed release characteristics to the extended release profile of a formulation.

(B) Drug-Layered Systems—that comprise an inert core and at least one drug-containing layer coated onto this core. The drug containing layer(s) may be further coated with a layer of a release rate controlling compound selected from those listed above. If the drug-containing layer of the drug-layered system does not contain any release rate controlling compounds and is of an immediate release nature, then a release rate controlling coating is necessary for achieving the modified profiles of the current invention.

In cases where the drug-containing layer is an extended release matrix layer described above, the release rate controlling coating is optional and allows for additional modification of the release profile. For example, the coating may be used to modulate the release (slow initially, faster later; or fast initially, slower later), or to provide a delay in the release. In particular, the release rate controlling coatings can include: cellulose esters, cellulose acetate, cellulose acetate butyrate, ethyl cellulose, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer, Type A), ethyl acrylate methyl methacrylate copolymer, polyvinyl acetate, cellulose acetate propionate, shellac, zein and combinations thereof.

In some embodiments of the invention, a core may not be inert but compositionally be of pure drug substance or a mixture of the drug substance and one or more pharmaceutically acceptable excipient producing an IR core. In such a case, the cores can undergo further processing as described above for inert cores to produce the desired extended release formulation.

Processes that may be used to produce formulations of this embodiment comprising a drug-containing core include solution or dry powder drug layering, compression coating, hot melt coating, supercritical fluid coating, electrostatic spray coating, agglomeration, granulation, pelletization, roller compaction, tablet compression, wet granulation with extrusion and spheronization, hot melt extrusion, and injection molding. Roller compaction, tablet compression, and the extrusion with spheronization processes are particularly helpful for the manufacturing of formulations with a high drug load.

Without putting any limitations thereon, exemplary formulations of the present invention having different modified pharmacokinetic (PK) profiles for methylergonovine are as follows.
(a) There may be mixed IR and XR particles in a capsule, compressed tablet or any other dosage form (IR/XR mixed particles). The IR particles provide the initial release of the therapeutic agent followed by extended release from the XR particles (IR/XR mixed population of particles).
(b) There may be a single population of particles in a capsule, compressed tablet or any other dosage form where the particles are either matrix XR particles, or IR cores further comprising an XR coating.
(c) There may be mixed particles in a capsule, compressed tablet or any other dosage form where XR particles of differing drug release characteristics are combined.
(d) There may be mixed particles in a capsule, compressed tablet or any other dosage form where delayed release (DR) particles of differing drug release characteristics are combined, optionally resulting in a pulsatile profile.
(e) There may be mixed particles m a capsule, compressed tablet or any other dosage form where IR particles are mixed with DR particles (IR/DR mixed particles). The IR particles provide the initial release of the therapeutic agent followed by release from the DR particles resulting in pulsed PK profiles (IR/DR mixed population of particles).
(f) There may be a single population of particles in a capsule, compressed tablet or any other dosage form where the pellet incorporates an IR core coated with DR coat, which is further coated with an IR drug layer. The outer IR drug layer provides an immediate release of the therapeutic agent followed by a delayed release from the DR core resulting in pulsed PK profile (IR/DR single population of particles).
(g) There may be mixed particles in a capsule, compressed tablet or any other dosage form where IR particles are mixed with DR coated XR particles (IR/DR-XR). The IR particles provide the initial release of the therapeutic agent followed by delayed and extended release from the DR coated XR particles (IR/DR-XR mixed population of particles).
(h) There may be a single population of particles in a capsule, compressed tablet or any other dosage form where the pellet incorporates an IR core coated with a XR coat, which is coated with a DR coat that is subsequently drug layered. The outer drug layer provides the initial immediate release of the therapeutic agent followed by delayed and extended release from the remainder of the pellet (IR/DR-XR single population of particles).
(i) There may be mixed particles in a capsule, compressed tablet or any other dosage form where XR particles are mixed with DR particles. The XR particles provide the initial and continuing release of the therapeutic agent followed by release from the DR particles (XR/DR mixed population of particles). A single population of particles in a capsule, compressed tablet or any other dosage form where the pellet incorporates an IR core coated with a DR coat which is then coated with a drug layer that is subsequently coated with an XR coat to produce a fast XR layer. The fast XR outer layer provides the initial release of the therapeutic agent followed by delayed release from the DR core (XR-f/DR single population of particles).
(j) There may be a XR tablet, which is either a matrix tablet or an XR-coated tablet.
(k) There may be a DR tablet coated with an IR drug layer.
(l) There may be one, or more than one, DR tablets mixed with one or more IR tablets in a capsule.
(m) There may be a XR tablet coated with a DR coat, then coated with an drug layer.
(n) There may be a bi-layer tablet with one layer containing the drug in XR form and a second layer containing the drug in an IR form.
(o) There may be a bi-layer tablet with one layer containing the drug in XR form and a second layer containing the drug in DR form.
(p) There may be a DR coated matrix tablet providing a DR/XR profile.

(C) Osmotic Release Systems—in a further embodiment, the invention provides an extended release methylergonovine preparation in the form of an osmotic tablet, wherein the drug release rate is determined by the rate of water permeation into the tablet core through a semi-permeable membrane coating.

For the preparation of an osmotic tablet, methylergonovine may be mixed with osmotic agent(s), tableting aides such as diluents and lubricants, and other commonly used excipients. The mixture is tableted either by direct compression or granulation followed by compression. Tablets are then coated with at least one release rate controlling compound that forms a semi-permeable membrane that surrounds each tablet.

The semipermeable membrane, which surrounds the drug-containing core, comprises at least one release rate controlling compound selected from cellulose esters, cellulose ethers, cellulose ester ethers and the like. Non-limiting examples of such compounds include cellulose acylate, cellulose ethyl ether, cellulose diacylate, cellulose triacylate, cellulose acetate, cellulose diacetate, cellulose triacetate, mono-, di- and tricellulose alkyls, mono-, di- and tricellulose aroyls, and combinations thereof. Additional release rate controlling compounds include ethyl cellulose, EUDRAGIT® NE 30 D (ethyl acrylate and methyl methacrylate copolymer), EUDRAGIT® RS (ammonio methacrylate copolymer, Type B) and EUDRAGIT® RL (ammonio methacrylate copolymer, Type A), ethyl acrylate methyl methacrylate copolymer, and combinations thereof.

The semi-permeable membrane may be applied on the tablets using standard coating techniques such as spraying, dipping, casting, coating solvent evaporation, molding or compression coating. An orifice is then drilled in the tablet coat using laser tablet drilling system or other mechanical means to allow the release of drug from the core.

Osmotic agents used for the practice of the current invention are well known in the art and include non-swellable compounds represented by, but not limited to polyols, carbohydrates (including monosaccharides, oligosaccharides, polysaccharides and sugar alcohols), acids, salts and hydrophilic compounds. For example, osmotic agents may be selected from mannitol, maltrin, xylitol, maltitol, lactitol, isomalt, sorbitol, arabitol, erythritol, ribitol, insositol, trehalose, lactose, glucose, sucrose, raffinose, fructose, dextran, glycine, urea, citric acid, tartaric acid, ascorbic acid, aspartame, malic acid, sodium chloride, potassium chloride, magnesium chloride, disodium hydrogen phosphate, sodium phosphate, potassium phosphate, sodium sulfate, lithium sulfate, magnesium sulfate, magnesium succinate, sodium bicarbonate, sodium carbonate, sodium acetate, sodium ascorbate, polyethylene glycol, maltodextrin, cyclodextrins and derivatives, non-swelling block polymers of PEO and PPO, polyethylene glycols, cellulose ethers, and combinations thereof.

Osmotic tablets can be formulated as a single or as a multiple layer core. In one embodiment, the osmotic tablet comprises a bilayer core, wherein one layer comprises agents to modulate drug release, such as a solubilizer, that are released in an extended manner, and the second layer comprises the drug and potentially other agents to modulate drug release.

An overcoat of drug can be applied to the tablet following a functional coating to provide an immediate release component to the dosage form. Alternatively, the osmotic tablet may be coated with an enteric compound on top of the semipermeable membrane providing a DR/XR profile.

In addition to the release rate control ling compounds, a number of pharmaceutically acceptable excipients may be used in the formulations of the invention as disclosed above. These excipients are well known in the art, and include binders and diluents, such as povidone, lactose, starch, gelatin, maltodextrin, methylcellulose, hydroxypropyl cellulose, hydroxypropyl methylcellulose, carboxymethylcellulose, sucrose, dextrose, acacia, tragacanth and locust bean gum, microcrystalline cellulose, dicalcium phosphate, calcium sulfate, cellulose, and talc; lubricants such as sodium stearyl fumarate and the metallic stearates such as magnesium stearate; wetting and solubilizing agents such as sodium docusate, sodium lauryl sulfate, polyethylene glycol, lecithin, poloxamer, polysorbates, polyoxyethylene ethers and sorbitan esters; disintegrants such as crosslinked sodium carboxymethylcellulose, sodium starch glycolate and crospovidone; buffering agents and/or pH modulating agents, such as aluminum hydroxide, ammonium bicarbonate, ammonium carbonate, ammonium phosphate, arginine, calcium acetate, calcium ascorbate, magnesium acetate, magnesium carbonate, potassium acetate, potassium bicarbonate, potassium carbonate, potassium phosphate dibasic, potassium sodium tartrate, potassium citrate, sodium citrate, sodium phosphate monobasic, sodium phosphate dibasic, sodium phosphate tribasic, sodium acetate, sodium bicarbonate, sodium ascorbate, sodium carbonate, fumaric acid, malic acid, tartaric acid, ascorbic acid, aspartic acid, alginic acid, glutamic acid, sorbic acid, and succinic acid; and glidants such as talc, starch and colloidal silicon dioxide; pore formers modulating the permeability of the semipermeable rate controlling membrane such as povidone, hypromellose, hydroxyethyl cellulose, hydroxypropyl cellulose, organic acids and salts amongst other excipients.

The amount of diluent in the modified release oral pharmaceutical composition of methylergonovine or a pharmaceutically acceptable salt thereof ranges from about 10% to about 60%, preferably from about 10% to about 50%, and more preferably from about 10% to about 40% by weight of the composition.

The amount of disintegrant in the modified release oral pharmaceutical composition of methylergonovine or a pharmaceutically acceptable salt thereof ranges from about 1% to about 50%, preferably from about 1% to about 30%, and more preferably from about 1% to about 15% by weight of the composition.

The amount of pH-modifying agent in the modified release oral pharmaceutical composition of methylergonovine or a pharmaceutically acceptable salt thereof ranges from about 0.1% to about 10%, preferably from about 0.1% to about 5%, and more preferably from about 0.1% to about 3% by weight of the composition.

The amount of glidant in the modified release oral pharmaceutical composition of methylergonovine or a pharmaceutically acceptable salt thereof ranges from about 0.1% to about 20%, and preferably from about 0.1% to about 10% by weight of the composition.

(D) Gastro-Retentive Systems—in a further embodiment, the invention provides an extended release methylergonovine preparation in the form of a gastro-retentive tablet, in particular a gastro-retentive extended release tablet. The gastro-retentive tablet is designed to be retained in the stomach for up to 6 hours after ingestion, after which the remaining dosage form and essentially all undissolved drug is released into the duodenum to transit through the gastrointestinal tract. The in-vitro dissolution profile used for the GR-ER tablet releases 80% of the dose of drug contained in the dosage form in approximately 10 hours.

### EXAMPLES

### Example 1

Composition of Methylergonovine Modified Release Matrix Tablet, 0.4 mg

| Item No. | Component | mg/Tablet |
|---|---|---|
| 1 | Methylergonovine Maleate, USP | 0.40 |
| 2 | Povidone | 3.00 |
| 3 | Gelatin | 1.00 |
| 4 | Acacia | 1.00 |
| 5 | Tartaric Acid | 0.60 |
| 6 | Methylparaben | 0.04 |
| 7 | Propylparaben | 0.01 |
| 8 | Corn Starch | 5.00 |
| 9 | Microcrystalline Cellulose | 20.00 |
| 10 | Lactose Monohydrate | 42.00 |
| 11 | Hypromellose (METHOCEL ®K15M) | 22.75 |
| 12 | Stearic Acid | 4.00 |
| 13 | Colloidal Silicon Dioxide | 0.20 |
| 14 | Purified Water, USP | qs |
| | Total | 100.00 |

Manufacturing Process:

The batch of Example 1 was manufactured by a wet granulation process. Lactose monohydrate, microcrystalline cellulose, corn starch, and povidone were dry mixed in a granulator and then was granulated with a solution containing methylergonovine maleate, gelatin, acacia, povidone, methylparaben and propylparaben dissolved in purified water. The wet granulation was oven dried at 40° C. to a moisture level of less than 1% (w/w) and then sized by passing through an 18 mesh sieve. The dried granules were then milled in a Fitzmill and blended with in a V-blender with hypromellose (METHOCEL® K15M), stearic acid and colloidal silicone dioxide. The final blend was compressed into modified-release tablets at the dose strength of 0.4 mg.

### Example 2

Composition of Methylergonovine Modified Release Matrix Tablet, 0.8 mg

| Item No. | Component | mg/Tablet |
|---|---|---|
| 1 | Methylergonovine Maleate, USP | 0.80 |
| 2 | Povidone | 3.00 |
| 3 | Gelatin | 1.00 |
| 4 | Acacia | 1.00 |
| 5 | Tartaric Acid | 1.60 |
| 6 | Methylparaben | 0.04 |
| 7 | Propylparaben | 0.01 |
| 8 | Corn Starch | 5.00 |
| 9 | Microcrystalline Cellulose | 20.00 |
| 10 | Lactose Monohydrate | 32.00 |
| 11 | Hypromellose (METHOCEL ®K15M) | 31.35 |
| 12 | Stearic Acid | 4.00 |
| 13 | Colloidal Silicon Dioxide | 0.20 |
| 14 | Purified Water, USP | qs |
| | Total | 100.00 |

Manufacturing Process:

The batch of Example 2 was manufactured by a wet granulation process. Lactose monohydrate, microcrystalline cellulose, corn starch, and povidone were dry mixed in a granulator and then was granulated with a solution containing methylergonovine maleate, gelatin, acacia, povidone, methylparaben and propylparaben dissolved in purified water. The wet granulation was oven dried at 40° C. to a moisture level of less than 1% (w/w) and then sized by passing through an 18 mesh sieve. The dried granules were then milled in Fitzmill and blended with in a V-blender with hypromellose (METHOCEL® K15M), stearic acid and colloidal silicone dioxide. The final blend was compressed into modified-release tablets at the dose strength of 0.4 mg.

### Example 3

Composition of Methylergonovine Immediate Release Matrix Tablet, 0.2 mg

| Item No. | Component | mg/Tablet |
|---|---|---|
| 1 | Methylergonovine Maleate, USP | 0.20 |
| 2 | Povidone | 3.00 |
| 3 | Gelatin | 1.00 |
| 4 | Acacia | 1.00 |
| 5 | Tartaric Acid | 0.60 |
| 6 | Methylparaben | 0.04 |
| 7 | Propylparaben | 0.01 |
| 8 | Corn Starch | 5.00 |
| 9 | Microcrystalline Cellulose | 43.15 |
| 10 | Lactose Monohydrate | 42.00 |
| 11 | Stearic Acid | 4.00 |
| 12 | Purified Water, USP | qs |
| | Total | 100.00 |

Manufacturing Process:

The batch of Example 3 was manufactured by a wet granulation process. Lactose monohydrate, microcrystalline cellulose, corn starch, and povidone were dry mixed in a granulator and then was granulated with a solution containing methylergonovine maleate, gelatin, acacia, povidone, methylparaben and propylparaben dissolved in purified water. The wet granulation was oven dried at 40° C. to a moisture level of less than 1% (w/w) and then sized by passing through an 18 mesh sieve. The dried granules were then milled in Fitzmill and blended within a V-blender with stearic acid to yield the immediate-release blend

### Example 4

Composition of Methylergonovine Bilayer Extended Release Matrix Tablet, 0.6 mg

The immediate release blend from Example 3 and the extended release blend from Example 1 were compressed into bilayer extended release tablets.

### Example 5

Composition of Immediate Release Methylergonovine Layered and Coated Beads

| Item No. | Component | mg/Capsule |
|---|---|---|
| 1 | Methylergonovine Maleate, USP | 0.20 |
| 2 | Povidone, USP | 3.00 |
| 3 | Gelatin, NF | 1.00 |
| 4 | Acacia, NF | 1.00 |
| 5 | Tartaric Acid, NF | 0.60 |
| 6 | Methylparaben, NF | 0.04 |
| 7 | Propylparaben, NF | 0.01 |
| 8 | Sugar Spheres, NF 30/35 mesh | 80.14 |
| 9 | Talc, USP | 4.00 |
| | Kollicoat ® | 10.00 |
| 10 | Purified Water, USP | qs |
| | Total | 100.00 |

Manufacturing Process:

The drug layering dispersion of Example 5 was prepared by dissolving the methylergonovine maleate, gelatin, acacia, povidone, methylparaben and propylparaben in purified water, then dispersing the talc, and stirring for 20 minutes. The drug dispersion was then applied onto the 30/35-mesh sugar spheres in a Glatt GPCG-1 fluidized bed coater while being stirred. The inlet temperature ranged between 50-55° C. to attain product at a temperature of 40-42° C. The dispersion was sprayed at the rate of 8-12 g/min with atomization air pressure of 1.5 bar. Optionally, the drug layered beads were subsequently applied with a polyvinyl alcohol-polyethylene glycol grafted copolymer (Kollicoat®) coating solution at 10% coating level and subsequently dried. The uncoated beads (100 mg) were filled into a capsule to yield immediate-release pellets in a capsule

### Example 6

Composition of Immediate Release Methylergonovine Microtablets in Capsule, 0.2 mg

| Item No. | Component | mg/Tablet |
|---|---|---|
| 1 | Methylergonovine Maleate, USP | 0.20 |
| 2 | Povidone, USP | 3.00 |
| 3 | Gelatin, NF | 1.00 |
| 4 | Acacia, NF | 1.00 |
| 5 | Tartaric Acid, NF | 0.60 |
| 6 | Methylparaben, NF | 0.04 |
| 7 | Propylparaben, NF | 0.01 |
| 8 | Lactose Monohydreate, NF | 42.00 |
| 9 | Microcrystalline Cellulose, NF | 43.15 |
| 10 | Corn Starch, NF | 5.00 |
| 11 | Stearic Acid, NF | 4.00 |
| 12 | Purified Water, USP | qs |
| | Total | 100.00 |

Manufacturing Process:

The drug layering solution of Example 6 was prepared by dissolving the methylergonovine maleate, gelatin, acacia, povidone, methylparaben and propylparaben in purified water. The drug solution was then sprayed onto a blend of lactose monohydrate, microcrystalline cellulose, corn starch and granulated using the wet granulation process. The wet granules were dried in an oven. The dried granules were them milled in Fitzmill and blended in a V-blender with stearic acid. The final blend was compressed into 2 mm microtablets using multipump tooling. Microtablets were subsequently coated with the polyvinyl alcohol-polyethylene glycol grafted copolymer (Kollicoat®) coating solution at 10% coating level and subsequently dried. 110 mg of these coated microtablets filled into the capsule to yield immediate release microtablets in a capsule.

### Example 7

Composition of Multiparticulate-Based, Modified Release Methylergonovine Capsule, 0.8 mg

The dried pellets from Example 5 were screened (stacked 20 mesh over 40 mesh sieves) and then seal coated using a Wurster process (GPCG-1) to a 5% weight gain with Opadry® II White. Following application of the seal coat, pellets were then coated on the GPCG-1 with Surelease® E-7-19010 to a weight gain of 15% (w/w). The coated pellets were oven cured for 24 hours at 50° C. Appropriate portions of extended release coated beads were then blended with immediate release beads from Example 5 and filled into hard gelatin capsules.

### Example 8

Composition of Microtablets-Based, Modified Release Methylergonovine Capsule, 0.6 mg

The compressed microtablets from Example 6 were seal coated using a Wurster process (GPCG-1) to 5% weight gain with Opadry® II White. Following application of the seal coat, pellets were then coated on the GPCG-1 with Surelease® E-7-19010 to a weight gain of 10% (w/w). The coated microtablets were oven cured for 24 hours at 50° C. Next, 300 mg of modified-release coated microtablets were then blended with immediate-release beads from Example 5 in appropriate portions and filled into hard gelatin capsules.

### Example 9

Composition of Multiparticulates-Based, Modified Release Methylergonovine Capsule

Immediate release beads from Example 5 were initially seal-coated with Hydroxypropyl Methylcellulose (Opadry®) at 6% weight gain and subsequently applied with dispersions of EUDRAGIT® RS (ammonio methacrylate copolymer Type B) and EUDRAGIT® RL (ammonio methacrylate copolymer Type A) polymers containing triethyl citrate and talc (anti-tacking agent) in various proportions and various coating levels and finally seal-coated with hydroxypropyl methylcellulose (Opadry®) at a 6% weight gain. The coated beads were encapsulated in hard gelatin capsules.

### Example 10

Twice Daily Tablets (Four Exemplary Structural Configurations)

In accordance with some embodiments, the present disclosure provides an oral modified release pharmaceutical composition suitable for twice daily administration comprising about 0.4 mg methylergonovine or a pharmaceutically acceptable salt thereof. Methylergonovine or a pharmaceutically acceptable salt thereof in the composition is released from the composition over a period ranging from about 0 hour to up to about 18 hours, for example, from about 0 hour to about 6 hours, or from 0 hour to about 12 hours.

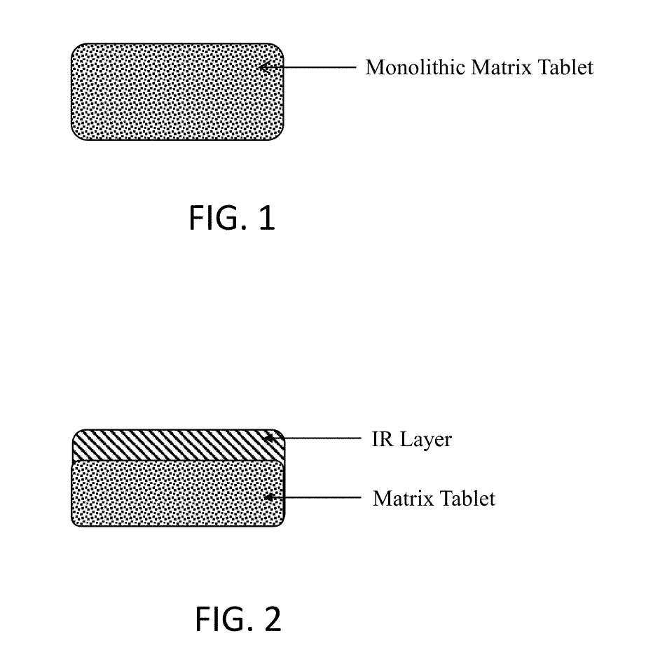

Tables 7-10 show four types of exemplary twice daily tablets having different structural configurations as shown in FIGS. 1-4, respectively. In the formulations described herein, “methylergometrine” is synonymous with “methylergonovine.” For brevity, Tables 7-10 include both twice daily tablets and once daily tablets as described herein (Example 11).

Referring to FIG. 1 and Table 7, an exemplary twice daily tablet is a monolithic matrix tablet containing 0.4 mg methylergonovine or a pharmaceutically acceptable salt thereof. Such a monolithic matrix tablet, which is an extended release (“ER”) formulation, is made as follows: Lactose monohydrate, microcrystalline cellulose, and maize starch are co-sifted through a suitable sieve to provide a blend. Separately, a granulating solution of methylergonovine maleate, povidone, and tartaric acid is prepared in purified water. The blend of lactose monohydrate, microcrystalline cellulose, and maize starch is mixed in a rapid mixer granulator, and then granulated with the granulating fluid. The wet mass is dried in a tray dryer at inlet 50° C. till its loss on drying (“LOD”) is no more than (“NMT”) 2.5% w/w at 105° C. The blend is dried and passed through a co-mill using a suitable screen. These granules are then blended with a sifted extragranular mixture comprising HPMC (hydroxypropyl methylcellulose), polyethylene oxide, sodium carboxymethylcellulose, Eudragits, hydroxypropyl cellulose NF, xanthan gum, ethylcellulose, and stearic acid in a blender. The resulting blend is compressed into tablets using suitable tooling with suitable physical parameters.

Referring to FIG. 2 and Table 8, an exemplary twice daily tablet is a bilayer tablet containing 0.4 mg (in total) methylergonovine or a pharmaceutically acceptable salt thereof. As illustrated in FIG. 2, such a bilayer tablet comprises an ER layer as a matrix, and an immediate release (“IR”) layer coated on the ER layer. In the manufacturing process for making such a bilayer tablet, the formulation for the ER Layer is first made following the procedure for the monolithic matrix tablet described above in FIG. 1 and Table 7, except the last step of compressing the blend into tablets.

Second, the formulation for the IR layer is made as follows: Lactose monohydrate, sodium starch glycolate, microcrystalline cellulose, and maize starch, are co-sifted through suitable sieve. Separately, a granulating solution of methylergonovine maleate, povidone, and tartaric acid, is prepared in purified water. The blend of lactose monohydrate, sodium starch glycolate, microcrystalline cellulose, and maize starch is mixed in a rapid mixer granulator, and is granulated with the granulating solution. The wet mass is dried in a tray dryer at inlet 50° C. till its LOD reaches no more than 2.5% w/w at 105° C. The blend is dried and passed through a co-mill using a suitable screen. These granules are blended with sifted stearic acid, which is an extragranular material in the IR layer, in a blender for form a blend for the IR layer.

Third, the lubricated blends for the ER layer and the IR layer are compressed into bilayer tablets using suitable tooling with suitable physical compression parameters.

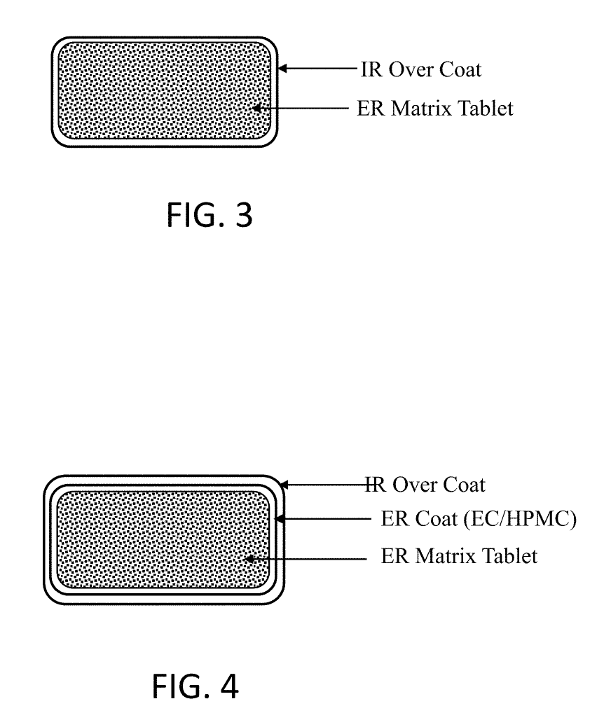

Referring to FIG. 3 and Table 9, an exemplary twice daily tablet includes an ER matrix tablet and an IR overcoat disposed on the ER matrix tablet, and contains 0.4 mg (in total) methylergonovine or a pharmaceutically acceptable salt thereof. In the manufacturing process for making such a tablet, first, the ER matrix tablet is made following the procedure for the monolithic matrix tablet described above in FIG. 1 and Table 7.

Second, a solution comprising methylergonovine maleate, povidone, and tartaric acid at a ratio shown in Table 9 is prepared in purified water with continuous stirring. Such a solution is then overcoated onto the compressed ER matrix tablet using suitable coating parameters.

Referring to FIG. 4 and Table 10, an exemplary twice daily tablet includes an ER matrix tablet, an ER coat disposed on the ER matrix tablet, and an IR overcoat disposed on the ER coat. As shown in Table 10, an exemplary twice daily tablet having the structural configuration as illustrated in FIG. 4 contains 0.4 mg (in total) methylergonovine or a pharmaceutically acceptable salt thereof. In the manufacturing process for making such a tablet, first, the ER matrix tablet is made following the procedure for the monolithic matrix tablet described above in FIG. 1 and Table 7.

Second, a solution for an ER coating and a solution for an IR overcoat are made. According to the formulations in Table 10, ethyl cellulose and HPMC of the required amount are weighed, and dissolved into a mixture of isopropyl alcohol USP and methylenechloride NF with continuous stirring to form a clear solution for the ER coating. Methylergonovine maleate, povidone, and tartaric acid are dissolved in purified water with continuous stirring to provide a solution for the IR overcoat.

Third, the ER coat and the IR overcoat are formed over the ER matrix tablet. The solution for the ER coat is coated onto the compressed ER matrix tablets using suitable coating parameters. The solution for the IR overcoat was then coated thereafter using suitable coating parameters. The resulting tablet structure is illustrated in FIG. 4.

TABLE 7
Monolithic Matrix Strategy

| | mg/Tab | | | | | | | | | | | | |
|---|---|---|---|---|---|---|---|---|---|---|---|---|---|
| | I- | I- | I- | I- | I- | I- | I- | I- | I- | I- | I- | I- | I- |
| Ingredients | Ex-1 | Ex-2 | Ex-3 | Ex-4 | Ex-5 | Ex-6 | Ex-7 | Ex-8 | Ex-9 | Ex-10 | Ex-11 | Ex-12 | Ex-13 |

Intragranular

| | | |
|---|---|---|
| 1 | Methylergometrine | D1 |
| | Maleate USP | |
| 2 | Lactose | 38 |
| | Monohydrate NF | |
| 3 | Microcrystalline | 33.7 |
| | Cellulose NF | |
| 4 | Maize Starch NF | 5 |
| 5 | Povidone LP K30 NF | 3 |
| 6 | Tartaric Acid NF | 0.6 |
| 7 | Purified water | q.s. |

Extragranular

| | | | | | | | | | | | | | | |
|---|---|---|---|---|---|---|---|---|---|---|---|---|---|---|
| 8 | Hypromellose | 40 | 20 | 60 | . . . | . . . | 20.0 | 20.0 | . . . | 20.0 | . . . | 25.0 | . . . | 15.0 |
| | USP (K100 M) | | | | | | | | | | | | | |
| 9 | Hypromellose | . . . | . . . | . . . | 40 | . . . | . . . | . . . | . . . | . . . | . . . | . . . | . . . | . . . |
| | USP (K4M) | | | | | | | | | | | | | |
| 10 | Polyethylene | . . . | . . . | . . . | . . . | 45.0 | 25.0 | . . . | . . . | . . . | . . . | . . . | . . . | . . . |
| | Oxide USP | | | | | | | | | | | | | |
| 11 | Sodium | . . . | . . . | . . . | . . . | . . . | . . . | 2.5.0 | 35.0 | 25.0 | . . . | . . . | . . . | . . . |
| | Carboxymethyl- | | | | | | | | | | | | | |
| | cellulose | | | | | | | | | | | | | |
| 12 | Hydroxypropyl | . . . | . . . | . . . | . . . | . . . | . . . | . . . | . . . | . . . | 40.0 | 30.0 | . . . | . . . |
| | cellulose NF | | | | | | | | | | | | | |
| 13 | Xanthan Gum | . . . | . . . | . . . | . . . | . . . | . . . | . . . | . . . | . . . | . . . | . . . | 30.0 | 25.0 |
| 14 | Eudragit | . . . | . . . | . . . | . . . | . . . | . . . | . . . | . . . | . . . | . . . | . . . | . . . | . . . |
| 15 | Ethylcellulose | . . . | . . . | . . . | . . . | . . . | . . . | . . . | . . . | . . . | . . . | . . . | . . . | . . . |
| 16 | Stearic Acid | 4 | 4 | 4 | 4 | 4 | 4 | 4 | 4 | 4 | 4 | 4 | 4 | 4 |

| | | | | | | | | | | | | | |
|---|---|---|---|---|---|---|---|---|---|---|---|---|---|
| TOTAL MATRIX | 125 | 105 | 145 | 125 | 130 | 130 | 130 | 120 | 130 | 125 | 140 | 115 | 125 |
| TAB WEIGHT | | | | | | | | | | | | | |

| | | mg/Tab | | | | | | | | Specification |
|---|---|---|---|---|---|---|---|---|---|---|
| | | I- | I- | I- | I- | I- | I- | I- | I- | range of total |
| Ingredients | | Ex-14 | Ex-15 | Ex-16 | Ex-17 | Ex-18 | Ex-19 | Ex-20 | Ex-21 | weight |
| Intragranular | | | | | | | | | | |
| 1 | Methylergometrine | D1 | | | | 0.6 | | | | 0.1%-1% |
| | Maleate USP | | | | | | | | | |
| 2 | Lactose | 38 | | | | 32.528 | | | | 10%-60% |
| | Monohydrate NF | | | | | | | | | |
| 3 | Microcrystalline | 33.7 | | | | 28.8472 | | | | 10%-60% |
| | Cellulose NF | | | | | | | | | |
| 4 | Maize Starch NF | 5 | | | | 4.28 | | | | 1%-50% |
| 5 | Povidone LP K30 NF | 3 | | | | 2.568 | | | | 0.5%-20% |
| 6 | Tartaric Acid NF | 0.6 | | | | 0.5136 | | | | 0.1%-10% |
| 7 | Purified water | q.s. | | | | q.s | | | | . . . |
| Extragranular | | | | | | | | | | |
| 8 | Hypromellose | . . . | 10.0 | . . . | . . . | 34.2 | 17.1 | 51.3 | . . . | 5%-95% |
| | USP (K100 M) | | | | | | | | | |
| 9 | Hypromellose | . . . | . . . | . . . | . . . | . . . | . . . | . . . | 34.2 | 5%-95% |
| | USP (K4M) | | | | | | | | | |
| 10 | Polyethylene | . . . | 15.0 | . . . | . . . | . . . | . . . | . . . | . . . | 5%-95% |
| | Oxide USP | | | | | | | | | |
| 11 | Sodium | . . . | . . . | . . . | . . . | . . . | . . . | . . . | . . . | 5%-95% |
| | Carboxymethyl- | | | | | | | | | |
| | cellulose | | | | | | | | | |
| 12 | Hydroxypropyl | . . . | . . . | . . . | . . . | . . . | . . . | . . . | . . . | 5%-95% |
| | cellulose NF | | | | | | | | | |
| 13 | Xanthan Gum | . . . | . . . | . . . | . . . | . . . | . . . | . . . | . . . | 5%-95% |
| 14 | Eudragit | 20.0 | 10.0 | . . . | 10.0 | . . . | . . . | . . . | . . . | 5%-95% |
| 15 | Ethylcellulose | . . . | . . . | 25.0 | 15.0 | . . . | . . . | . . . | . . . | 5%-95% |
| 16 | Stearic Acid | 4 | 4 | 4 | 4 | 3.4 | 3.4 | 3.4 | 3.4 | 0.1%-20% |

| | | | | | | | | | |
|---|---|---|---|---|---|---|---|---|---|
| TOTAL MATRIX | 105 | 120 | 110 | 110 | 107 | 90 | 124 | 107 | . . . |
| TAB WEIGHT | | | | | | | | | |

D1 is in a range from 0.4 mg to 0.7 mg (e.g., 0.4 mg, 0.5 mg, 0.6 mg, 0.7 mg or fractional values between these stated values) of Methylergometrine Maleate USP.

TABLE 8
Bilayer Tablet (IR-ER)

| | | | | | | | | | | |
|---|---|---|---|---|---|---|---|---|---|---|
| II- | II- | II- | II- | II- | II- | II- | II- | II- | II- | II- |
| Ex-1 | Ex-2 | Ex-3 | Ex-4 | Ex-5 | Ex-6 | Ex-7 | Ex-8 | Ex-9 | Ex-10 | Ex-11 |

A. IMMEDIATE RELEASE LAYER
Intragranular

| | | |
|---|---|---|
| 1 | Methylergometrine | 0.100 |
| | Maleate USP | |
| 2 | Microcrystalline | 10.000 |
| | Cellulose NF | |
| 3 | Lactose | 15.100 |
| | Monohydrate NF | |
| 4 | Sodium Starch | 1.500 |
| | Glycolate | |
| 5 | Maize Starch NF | 2.500 |
| 6 | Tartaric Acid NF | 0.300 |
| 7 | Purified water | q.s. |

Extragranular

| | | |
|---|---|---|
| 8 | Stearic Acid | 0.500 |

| | |
|---|---|
| Total IR Layer Weight | 30.000 |

B. EXTENDED RELEASE LAYER
Intragranular

| | | |
|---|---|---|
| 9 | Methylergometrine | D2 |
| | Maleate USP | |
| 10 | Lactose | 32.53 |
| | Monohydrate NF | |
| 11 | Microcrystalline | 28.85 |
| | Cellulose NF | |
| 12 | Maize Starch NF | 4.28 |
| 13 | Povidone LP K30 NF | 2.57 |
| 14 | Tartaric Acid NF | 0.51 |
| 15 | Purified water | q.s |

Extragranular

| | | | | | | | | | | | | |
|---|---|---|---|---|---|---|---|---|---|---|---|---|
| 16 | Hypromellose USP | 34.24 | 17.14 | 51.31 | . . . | . . . | 17.1 | 17.1 | . . . | 17.1 | . . . | 21.4 |
| | (K100 M) | | | | | | | | | | | |
| 17 | Hypromellose USP | . . . | . . . | . . . | 34 | . . . | . . . | . . . | . . . | . . . | . . . | . . . |
| | (K4M) | | | | | | | | | | | |
| 18 | Polyethylene Oxide | . . . | . . . | . . . | . . . | 38.6 | 21.4 | . . . | . . . | . . . | . . . | . . . |
| | USP | | | | | | | | | | | |
| 19 | Sodium | . . . | . . . | . . . | . . . | . . . | . . . | 21.4 | 30.0 | 21.4 | . . . | . . . |
| | Carboxymethyl- | | | | | | | | | | | |
| | cellulose | | | | | | | | | | | |
| 20 | Hydroxypropyl | . . . | . . . | . . . | . . . | . . . | . . . | . . . | . . . | . . . | 34.3 | 25.7 |
| | cellulose NF | | | | | | | | | | | |
| 21 | Xanthan Gum | . . . | . . . | . . . | . . . | . . . | . . . | . . . | . . . | . . . | . . . | . . . |
| 22 | Eudragit | . . . | . . . | . . . | . . . | . . . | . . . | . . . | . . . | . . . | . . . | . . . |
| 23 | Ethylcellulose | . . . | . . . | . . . | . . . | . . . | . . . | . . . | . . . | . . . | . . . | . . . |
| 24 | Stearic Acid | 3.4 | 3.4 | 3.4 | 3.4 | 3.4 | 3.4 | 3.4 | 3.4 | 3.4 | 3.4 | 3.4 |

| | | | | | | | | | | | |
|---|---|---|---|---|---|---|---|---|---|---|---|
| Total ER Layer Weight | 107 | 90 | 124 | 107 | 111 | 111.43 | 111.43 | 102.86 | 111.43 | 107.14 | 120 |
| TOTAL BILAYER | 137 | 120 | 154 | 137 | 141.4 | 141.4 | 141.4 | 132.9 | 141.4 | 137.1 | 150 |
| TAB WEIGHT | | | | | | | | | | | |

| | | | | | | | | Specification |
|---|---|---|---|---|---|---|---|---|
| | | II- | II- | II- | II- | II- | II- | range of total |
| | | Ex-12 | Ex-13 | Ex-14 | Ex-15 | Ex-16 | Ex-17 | weight |
| A. IMMEDIATE RELEASE LAYER | | | | | | | | |
| Intragranular | | | | | | | | |
| 1 | Methylergometrine | 0.100 | | | | | | 0.01%-1% |
| | Maleate USP | | | | | | | |
| 2 | Microcrystalline | 10.000 | | | | | | 1%-50% |
| | Cellulose NF | | | | | | | |
| 3 | Lactose | 15.100 | | | | | | 1%-50% |
| | Monohydrate NF | | | | | | | |
| 4 | Sodium Starch | 1.500 | | | | | | 0.1%-20% |
| | Glycolate | | | | | | | |
| 5 | Maize Starch NF | 2.500 | | | | | | 0.1%-20% |
| 6 | Tartaric Acid NF | 0.300 | | | | | | 0.01%-20% |
| 7 | Purified water | q.s. | | | | | | . . . |
| Extragranular | | | | | | | | |
| 8 | Stearic Acid | 0.500 | | | | | | . . . |
| Total IR Layer Weight | | 30.000 | | | | | | . . . |
| B. EXTENDED RELEASE LAYER | | | | | | | | |
| Intragranular | | | | | | | | |
| 9 | Methylergometrine | D2 | | | | | | 0.1%-1% |
| | Maleate USP | | | | | | | |
| 10 | Lactose | 32.53 | | | | | | 10%-60% |
| | Monohydrate NF | | | | | | | |
| 11 | Microcrystalline | 28.85 | | | | | | 10%-60% |
| | Cellulose NF | | | | | | | |
| 12 | Maize Starch NF | 4.28 | | | | | | 1%-50% |
| 13 | Povidone LP K30 NF | 2.57 | | | | | | 0.5%-20% |
| 14 | Tartaric Acid NF | 0.51 | | | | | | 0.1%-10% |
| 15 | Purified water | q.s | | | | | | . . . |
| Extragranular | | | | | | | | |
| 16 | Hypromellose USP | . . . | 12.9 | . . . | 8.6 | . . . | . . . | 5%-95% |
| | (K100 M) | | | | | | | |
| 17 | Hypromellose USP | . . . | . . . | . . . | . . . | . . . | . . . | 5%-95% |
| | (K4M) | | | | | | | |
| 18 | Polyethylene Oxide | . . . | . . . | . . . | 12.9 | . . . | . . . | 5%-95% |
| | USP | | | | | | | |
| 19 | Sodium | . . . | . . . | . . . | . . . | . . . | . . . | 5%-95% |
| | Carboxymethyl- | | | | | | | |
| | cellulose | | | | | | | |
| 20 | Hydroxypropyl | . . . | . . . | . . . | . . . | . . . | . . . | 5%-95% |
| | cellulose NF | | | | | | | |
| 21 | Xanthan Gum | 25.7 | 21.4 | . . . | . . . | . . . | . . . | 5%-95% |
| 22 | Eudragit | . . . | . . . | 17.1 | 8.6 | . . . | 8.6 | 5%-95% |
| 23 | Ethylcellulose | . . . | . . . | . . . | . . . | 21.4 | 12.9 | 5%-95% |
| 24 | Stearic Acid | 3.4 | 3.4 | 3.4 | 3.4 | 3.4 | 3.4 | 0.1%-20% |

| | | | | | | | |
|---|---|---|---|---|---|---|---|
| Total ER Layer Weight | 98.57 | 107.14 | 90 | 102.86 | 94.286 | 94.286 | . . . |
| TOTAL BILAYER | 128.6 | 137.1 | 120 | 132.9 | 124.3 | 124.3 | . . . |
| TAB WEIGHT | | | | | | | |

D2 is in a range from 0.3 mg to 0.6 mg (e.g., 0.3 mg, 0.4 mg, 0.5 mg, 0.6 mg or fractional values between these stated values) of Methylergometrine Maleatc USP.

TABLE 9
IR Over Coat on ER Matrix

| | | | | | | | | | | |
|---|---|---|---|---|---|---|---|---|---|---|
| III- | III- | III- | III- | III- | III- | III- | III- | III- | III- | III- |
| Ex-1 | Ex-2 | Ex-3 | Ex-4 | Ex-5 | Ex-6 | Ex-7 | Ex-8 | Ex-9 | Ex-10 | Ex-11 |

A. EXTENDED RELEASE MATRIX CORE
Intragranular

| | | |
|---|---|---|
| 1 | Methylergometrine | D3 |
| | Maleate USP | |
| 2 | Lactose Monohydrate | 32.53 |
| | NF | |
| 3 | Microcrystalline | 28.85 |
| | Cellulose NF | |
| 4 | Maize Starch NF | 4.28 |
| 5 | Povidone LP K30 NF | 2.57 |
| 6 | Tartaric Acid NF | 0.51 |
| 7 | Purified water | q.s |

Extragranular

| | | | | | | | | | | | | |
|---|---|---|---|---|---|---|---|---|---|---|---|---|
| 8 | Hypromellose USP | 34.24 | 17.14 | 51.31 | . . . | . . . | 17.1 | 17.1 | . . . | 17.1 | . . . | 21.4 |
| | (K100 M) | | | | | | | | | | | |
| 9 | Hypromellose USP | . . . | . . . | . . . | 34 | . . . | . . . | . . . | . . . | . . . | . . . | . . . |
| | (K4M) | | | | | | | | | | | |
| 10 | Polyethylene Oxide | . . . | . . . | . . . | . . . | 38.6 | 21.4 | . . . | . . . | . . . | . . . | . . . |
| | USP | | | | | | | | | | | |
| 11 | Sodium | . . . | . . . | . . . | . . . | . . . | . . . | 21.4 | 30.0 | 21.4 | . . . | . . . |
| | Carboxymethyl- | | | | | | | | | | | |
| | cellulose | | | | | | | | | | | |
| 12 | Hydroxypropyl | . . . | . . . | . . . | . . . | . . . | . . . | . . . | . . . | . . . | 34.3 | 25.7 |
| | cellulose NF | | | | | | | | | | | |
| 13 | Xanthan Gum | . . . | . . . | . . . | . . . | . . . | . . . | . . . | . . . | . . . | . . . | . . . |
| 14 | Eudragit | . . . | . . . | . . . | . . . | . . . | . . . | . . . | . . . | . . . | . . . | . . . |
| 15 | Ethylcellulose | . . . | . . . | . . . | . . . | . . . | . . . | . . . | . . . | . . . | . . . | . . . |
| 16 | Stearic Acid | 3.4 | 3.4 | 3.4 | 3.4 | 3.4 | 3.4 | 3.4 | 3.4 | 3.4 | 3.4 | 3.4 |

| | | | | | | | | | | | |
|---|---|---|---|---|---|---|---|---|---|---|---|
| Total Matrix Tablet Weight | 107 | 90 | 124 | 107 | 111 | 111 | 111 | 103 | 111 | 107 | 120 |

B. IMMEDIATE RELEASE LAYER

| | | |
|---|---|---|
| 17 | Methylergometrine | 0.100 |
| | Maleate USP | |
| 18 | Povidone LP K30 NF | 5.000 |
| 19 | Tartaric Acid NF | 0.300 |
| 20 | Purified water | q.s. |

| Total IR Layer Weight | 5.4 | | | | | | | | | | |
|---|---|---|---|---|---|---|---|---|---|---|---|
| TOTAL TAB WEIGHT | 112 | 95 | 129 | 112 | 117 | 117 | 117 | 108 | 117 | 113 | 125 |

| | | | | | | | | Specification |
|---|---|---|---|---|---|---|---|---|
| | | III- | III- | III- | III- | III- | III- | range of total |
| | | Ex-12 | Ex-13 | Ex-14 | Ex-15 | Ex-16 | Ex-17 | weight |
| A. EXTENDED RELEASE MATRIX CORE | | | | | | | | |
| Intragranular | | | | | | | | |
| 1 | Methylergometrine | D3 | | | | | | 0.1%-1% |
| | Maleate USP | | | | | | | |
| 2 | Lactose Monohydrate | 32.53 | | | | | | 10%-60% |
| | NF | | | | | | | |
| 3 | Microcrystalline | 28.85 | | | | | | 10%-60% |
| | Cellulose NF | | | | | | | |
| 4 | Maize Starch NF | 4.28 | | | | | | 1%-50% |
| 5 | Povidone LP K30 NF | 2.57 | | | | | | 0.5%-20% |
| 6 | Tartaric Acid NF | 0.51 | | | | | | 0.1%-10% |
| 7 | Purified water | q.s | | | | | | . . . |
| Extragranular | | | | | | | | |
| 8 | Hypromellose USP | . . . | 12.9 | . . . | 8.6 | . . . | . . . | 5%-95% |
| | (K100 M) | | | | | | | |
| 9 | Hypromellose USP | . . . | . . . | . . . | | . . . | . . . | 5%-95% |
| | (K4M) | | | | | | | |
| 10 | Polyethylene Oxide | . . . | . . . | . . . | 12.9 | . . . | . . . | 5%-95% |
| | USP | | | | | | | |
| 11 | Sodium | . . . | . . . | . . . | . . . | . . . | . . . | 5%-95% |
| | Carboxymethyl- | | | | | | | |
| | cellulose | | | | | | | |
| 12 | Hydroxypropyl | . . . | . . . | . . . | . . . | . . . | . . . | 5%-95% |
| | cellulose NF | | | | | | | |
| 13 | Xanthan Gum | 25.7 | 21.4 | . . . | . . . | . . . | . . . | 5%-95% |
| 14 | Eudragit | . . . | . . . | 17.1 | 8.6 | . . . | 8.6 | 5%-95% |
| 15 | Ethylcellulose | . . . | . . . | . . . | . . . | 21.4 | 12.9 | 5%-95% |
| 16 | Stearic Acid | 3.4 | 3.4 | 3.4 | 3.4 | 3.4 | 3.4 | 0.1%-20% |

| | | | | | | | |
|---|---|---|---|---|---|---|---|
| Total Matrix Tablet Weight | 98.6 | 107 | 90 | 103 | 94.3 | 94.3 | . . . |

| B. IMMEDIATE RELEASE LAYER | | | |
|---|---|---|---|
| 17 | Methylergometrine | 0.100 | 0.01%-1% |
| | Maleate USP | | |
| 18 | Povidone LP K30 NF | 5.000 | 0.1%-20% |
| 19 | Tartaric Acid NF | 0.300 | 0.01%-20% |
| 20 | Purified water | q.s. | . . . |

| Total IR Layer Weight | 5.4 | | | | | | . . . |
|---|---|---|---|---|---|---|---|
| TOTAL TAB WEIGHT | 104 | 113 | 95 | 108 | 100 | 100 | . . . |

D3 is in the range from 0.3 mg to 0.6 mg (e.g., 0.3 mg, 0.4 mg, 0.5 mg, 0.6 mg or fractional values between these stated values) of Methylergometrine Maleate USP.

TABLE 10
IR over coat -ER coat on ER matrix

| | | | | | | | | | | |
|---|---|---|---|---|---|---|---|---|---|---|
| IV- | IV- | IV- | IV- | IV- | IV- | IV- | IV- | IV- | IV- | IV- |
| Ex-1 | Ex-2 | Ex-3 | Ex-4 | Ex-5 | Ex-6 | Ex-7 | Ex-8 | Ex-9 | Ex-10 | Ex-11 |

A. EXTENDED RELEASE MATRIX CORE
Intragranular

| | | |
|---|---|---|
| 1 | Methylergometrine | D4 |
| | Maleate USP | |
| 2 | Lactose | 32.53 |
| | Monohydrate NF | |
| 3 | Macrocrystalline | 28.85 |
| | Cellulose NF | |
| 4 | Maize Starch NF | 4.28 |
| 5 | Povidone LP K30 NF | 2.57 |
| 6 | Tartaric Acid NF | 0.51 |
| 7 | Purified water | q.s |

Extragranular

| | | | | | | | | | | | | |
|---|---|---|---|---|---|---|---|---|---|---|---|---|
| 8 | Hypromellose USP | 34.24 | 17.14 | 51.31 | . . . | . . . | 17.1 | 17.1 | . . . | 17.1 | . . . | 21.4 |
| | (K100 M) | | | | | | | | | | | |
| 9 | Hypromellose USP | . . . | . . . | . . . | 34 | . . . | . . . | . . . | . . . | . . . | . . . | . . . |
| | (K4M) | | | | | | | | | | | |
| 10 | Polyethylene Oxide | . . . | . . . | . . . | . . . | 38.6 | 21.4 | . . . | . . . | . . . | . . . | . . . |
| | USP | | | | | | | | | | | |
| 11 | Sodium | . . . | . . . | . . . | . . . | . . . | . . . | 21.4 | 30.0 | 21.4 | . . . | . . . |
| | Carboxymethyl- | | | | | | | | | | | |
| | cellulose | | | | | | | | | | | |
| 12 | Hydroxypropyl | . . . | . . . | . . . | . . . | . . . | . . . | . . . | . . . | . . . | 34.3 | 25.7 |
| | cellulose NF | | | | | | | | | | | |
| 13 | Xanthan Gum | . . . | . . . | . . . | . . . | . . . | . . . | . . . | . . . | . . . | . . . | . . . |
| 14 | Eudragit | . . . | . . . | . . . | . . . | . . . | . . . | . . . | . . . | . . . | . . . | . . . |
| 15 | Ethylcellulose | . . . | . . . | . . . | . . . | . . . | . . . | . . . | . . . | . . . | . . . | . . . |
| 16 | Stearic Acid | 3.4 | 3.4 | 3.4 | 3.4 | 3.4 | 3.4 | 3.4 | 3.4 | 3.4 | 3.4 | 3.4 |

| | | | | | | | | | | | |
|---|---|---|---|---|---|---|---|---|---|---|---|
| Total Matrix Tablet weight | 107 | 90 | 124 | 107 | 111 | 111 | 111 | 103 | 111 | 107.14 | 120 |

B. EXTENDED RELEASE COATING

| | | |
|---|---|---|
| 1 | Ethyl cellulose | 4.000 |
| | (7/10 cps) | |
| 2 | Hypromellose USP | 3.000 |
| | (3/5/10 cps) | |
| 3 | Ispropyl alcohol | q.s |
| 4 | Methylene chloride | q.s |

| | |
|---|---|
| Total ER Coat weight | 7 |

C. IMMEDIATE RELEASE OVER COAT

| | | |
|---|---|---|
| 5 | Methylergometrine | 0.100 |
| | Maleate USP | |
| 6 | Povidone LP K30 NF | 5.000 |
| 7 | Tartaric Acid NF | 0.300 |
| 8 | Purified water | q.s |

| Tolal IR over coat Weight | 5.400 | | | | | | | | | | |
|---|---|---|---|---|---|---|---|---|---|---|---|
| TOTAL TAB WEIGHT | 119 | 102 | 136 | 119 | 124 | 124 | 124 | 115 | 124 | 120 | 132 |

| | | | | | | | | Specification |
|---|---|---|---|---|---|---|---|---|
| | | IV- | IV- | IV- | IV- | IV- | IV- | range of total |
| | | Ex-12 | Ex-13 | Ex-14 | Ex-15 | Ex-16 | Ex-17 | weight |
| A. EXTENDED RELEASE MATRIX CORE | | | | | | | | |
| Intragranular | | | | | | | | |
| 1 | Methylergometrine | D4 | | | | | | 0.1%-1% |
| | Maleate USP | | | | | | | |
| 2 | Lactose | 32.53 | | | | | | 10%-60% |
| | Monohydrate NF | | | | | | | |
| 3 | Macrocrystalline | 28.85 | | | | | | 10%-60% |
| | Cellulose NF | | | | | | | |
| 4 | Maize Starch NF | 4.28 | | | | | | 1%-50% |
| 5 | Povidone LP K30 NF | 2.57 | | | | | | 0.5%-20% |
| 6 | Tartaric Acid NF | 0.51 | | | | | | 0.1%-10% |
| 7 | Purified water | q.s | | | | | | . . . |
| Extragranular | | | | | | | | |
| 8 | Hypromellose USP | . . . | 12.9 | . . . | 8.6 | . . . | . . . | 5%-95% |
| | (K100 M) | | | | | | | |
| 9 | Hypromellose USP | . . . | . . . | . . . | | . . . | . . . | 5%-95% |
| | (K4M) | | | | | | | |
| 10 | Polyethylene Oxide | . . . | . . . | . . . | 12.9 | . . . | . . . | 5%-95% |
| | USP | | | | | | | |
| 11 | Sodium | . . . | . . . | . . . | . . . | . . . | . . . | 5%-95% |
| | Carboxymethyl- | | | | | | | |
| | cellulose | | | | | | | |
| 12 | Hydroxypropyl | . . . | . . . | . . . | . . . | . . . | . . . | 5%-95% |
| | cellulose NF | | | | | | | |
| 13 | Xanthan Gum | 25.7 | 21.4 | . . . | . . . | . . . | . . . | 5%-95% |
| 14 | Eudragit | . . . | . . . | 17.1 | 8.6 | . . . | 8.6 | 5%-95% |
| 15 | Ethylcellulose | . . . | . . . | . . . | . . . | 21.4 | 12.9 | 5%-95% |
| 16 | Stearic Acid | 3.4 | 3.4 | 3.4 | 3.4 | 3.4 | 3.4 | 0.1%-20% |

| | | | | | | | |
|---|---|---|---|---|---|---|---|
| Total Matrix Tablet weight | 98.57 | 107.14 | 90 | 102.86 | 94.29 | 94.29 | . . . |

| B. EXTENDED RELEASE COATING | | | |
|---|---|---|---|
| 1 | Ethyl cellulose | 4.000 | 0.1%-20% |
| | (7/10 cps) | | |
| 2 | Hypromellose USP | 3.000 | 0.1%-20% |
| | (3/5/10 cps) | | |
| 3 | Ispropyl alcohol | q.s | . . . |
| 4 | Methylene chloride | q.s | . . . |
| Total ER Coat weight | | 7 | . . . |
| C. IMMEDIATE RELEASE OVER COAT | | | |
| 5 | Methylergometrine | 0.100 | 0.01%-1% |
| | Maleate USP | | |
| 6 | Povidone LP K30 NF | 5.000 | 0.1%-20% |
| 7 | Tartaric Acid NF | 0.300 | 0.01%-20% |
| 8 | Purified water | q.s | . . . |

| Tolal IR over coat Weight | 5.400 | | | | | | . . . |
|---|---|---|---|---|---|---|---|
| TOTAL TAB WEIGHT | 111 | 120 | 102 | 115 | 107 | 107 | . . . |

D4 is in a range from 0.3 mg to 0.6 mg (e.g., 0.3 mg, 0.4 mg, 0.5 mg, 0.6 mg or fractional values between these stated values) of Methylergometrine Maleate USP.

### Example 11

Once Daily Tablets (Four Exemplary Structural Configurations)

In accordance with some embodiments, the present disclosure provides an oral modified release pharmaceutical composition suitable for once daily administration comprising about 0.7 mg in total of methylergonovine or a pharmaceutically acceptable salt thereof.

Tables 7-10 show four types of exemplary once daily tablets having different structural configurations as shown in FIGS. 1-4, respectively.

Referring to Table 7, each of the exemplary once daily tablets can be a monolithic matrix tablet having a structure as illustrated in FIG. 1, and containing 0.6 mg or 0.7 mg methylergonovine or a pharmaceutically acceptable salt thereof. The once daily tablets are made using the procedures described above as for the twice daily tablets in Table 7.

Referring to Table 8, each of the exemplary once daily tablets can be a bilayer tablet having a structure as illustrated in FIG. 2, and containing 0.7 mg methylergonovine or a pharmaceutically acceptable salt thereof. The once daily tablets are made using the procedures as described as for the twice daily tablets in Table 8.

Referring to Table 9, each of the exemplary once daily tablets includes an ER matrix tablet and an IR overcoat disposed on the ER matrix tablet as illustrated in FIG. 3, and can contain 0.7 mg (in total) methylergonovine or a pharmaceutically acceptable salt thereof. The once daily tablets are made using the procedures as described as for the twice daily tablets in Table 9.

Referring to Table 10, each of the exemplary once daily tablets has a structure as illustrated in FIG. 4, and includes an ER matrix tablet, an ER coat disposed on the ER matrix tablet, and an IR overcoat disposed on the ER coat. As shown in Table 10, each exemplary once daily tablet contains 0.7 mg (in total) methylergonovine or a pharmaceutically acceptable salt thereof. The once daily tablets are made using the procedures as described as for the twice daily tablets in Table 10.

### Example 12

Pulse Release Formulations and Resulting Capsule or Tablet

In accordance with some embodiments, the present disclosure provides a pulse release composition comprising methylergonovine or a pharmaceutically acceptable salt thereof in the range of from about 0.4 mg to about 0.7 mg, which is suitable for twice daily or once daily oral administration.

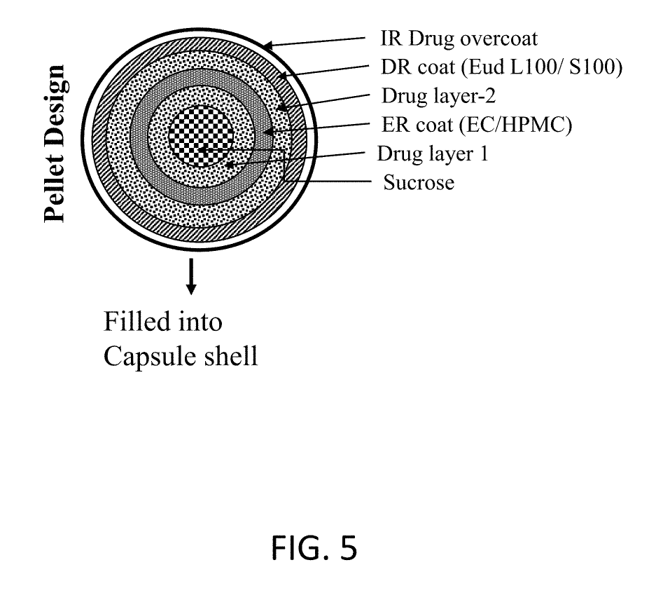

Tables 11-13 show three types of exemplary capsules or tablets having different structural configurations as shown in FIGS. 5-7, respectively.

Referring to FIG. 5 and Table 11, each of the exemplary capsules o pulse release pellets filled in HPMC or hard gelatin capsule. The pulse release pellets have a structure as illustrated in FIG. 5, which comprises at least five layers including a first drug layer (“Drug layer-1”), an extended release (“ER”) coat, a second drug layer (“Drug layer-2”), a delay release (“DR”) coat, and an immediate release (“IR”) overcoat comprising the active drug ingredient. The formulations for each layer are shown in Table 11. Each of the pulse release pellets may optionally comprise a sucrose core as shown in FIG. 5. Such a pulse release capsule contains methylergonovine maleate in the range of from about 0.4 mg to about 0.7 mg in total, for example, 0.4 mg, 0.45 mg, 0.6 mg, and 0.7 mg. The capsules containing about 0.4 mg or about 0.45 mg of methylergonovine maleate are suitable for twice daily (“BID”) administration, and the capsules containing about 0.6 mg or about 0.7 mg of methylergonovine maleate are suitable for once daily (“OD”) administration.

The pulse release pellets and capsules in Table 11 are made as follows:

Step 3.1. Preparation of Drug Layer-1: According to the formulation for Drug Layer-1 in Table 11, methylergonovine maleate, povidone, tartaric acid are dissolved in purified water to provide a first dispersion solution. Sugar (sucrose) beads are sifted from sieves of 30/35 mesh. The dispersion is sprayed onto the sugar beads in a Glatt GPCG-1 fluidised bed coater while being stirred under suitable experimental conditions.

Step 3.2, Formation of the ER Coat: According to the formulation for the ER coat in Table 11, Ethyl cellulose and HPMC are dissolved in a mixture of methylene chloride and isopropyl alcohol to provide a solution of the ER coat. The beads having the first drug layer are coated with the solution for the ER coat to obtain required a weight gain.

Step 3.3. Coating of Drug Layer-2: According to the formulation for Drug Layer-2 in Table 11, methylergonovine maleate, povidone, and tartaric acid are dissolved in purified water to provide a second dispersion solution. The second dispersion is sprayed onto the coated beads from step 3.2 in a Glatt GPCG-1 fluidised bed coater while being stirring under suitable experimental conditions.

Step 3.4. Coating of the DR Coat: According to the formulation for the DR coat in Table 11, Eudragit L100, Eudragit S100, and dibutyl sebacate are dissolved in a mixture of acetone, isopropyl alcohol and water to provide a solution for the DR coat. The main ingredient of Eudragit L100 is a copolymer of methacrylic acid and methyl methacrylate (1:1). The main ingredient of Eudragit S100 is a copolymer of methacrylic acid and methyl methacrylate (1:2). The beads having the second drug layer are coated with the solution for the DR coat to obtain required a weight gain.

Step 3.5. Coating of the IR Overcoat: According to the formulation for the IR overcoat in Table 11, methylergonovine maleate, povidone, and tartaric acid are dissolved in purified water to provide a third dispersion solution. The third dispersion solution is sprayed onto the coated beads from step 3.4 in a Glatt GPCG-1 fluidised bed coater while being stirring under suitable experimental conditions to provide the pulse release pellets having the structure illustrated in FIG. 5.

Step 3.6. Capsule filling: The pulse release pellets are filled into capsules comprising HPMC or hard gelatin. Stearic acid is used as a lubricant.

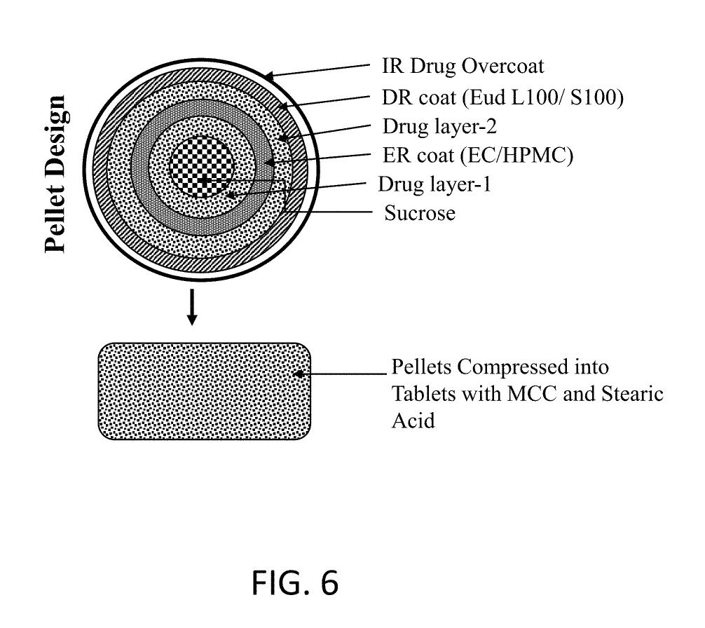

Referring to FIG. 6 and Table 12, each of the exemplary tablets comprises pulse release pellets compressed in a lubricated blend comprising microcrystalline cellulose NF and stearic acid. The pulse release pellets have the same structure as that illustrated in FIG. 5, and each comprises at least five layers including a first drug layer (“Drug layer-1”), an ER coat, a second drug layer (“Drug layer-2”), a DR coat, and an IR overcoat. The formulations for each layer are shown in Table 12. The tablets containing about 0.4 mg or about 0.45 mg of methylergonovine maleate are suitable for twice daily administration, and the capsules containing about 0.6 mg or about 0.7 mg of methylergonovine maleate are suitable for once daily administration. The pulse release pellets are manufactured following the procedures, steps 3.1-3.5, as described above. The pulse release pellets are mixed with sifted microcrystalline cellulose and stearic acid for 15-20 minutes to form a lubricated blend. The lubricated blend is then compressed using suitable tooling and physical parameters to form pulse release tablets as illustrated in FIG. 6.

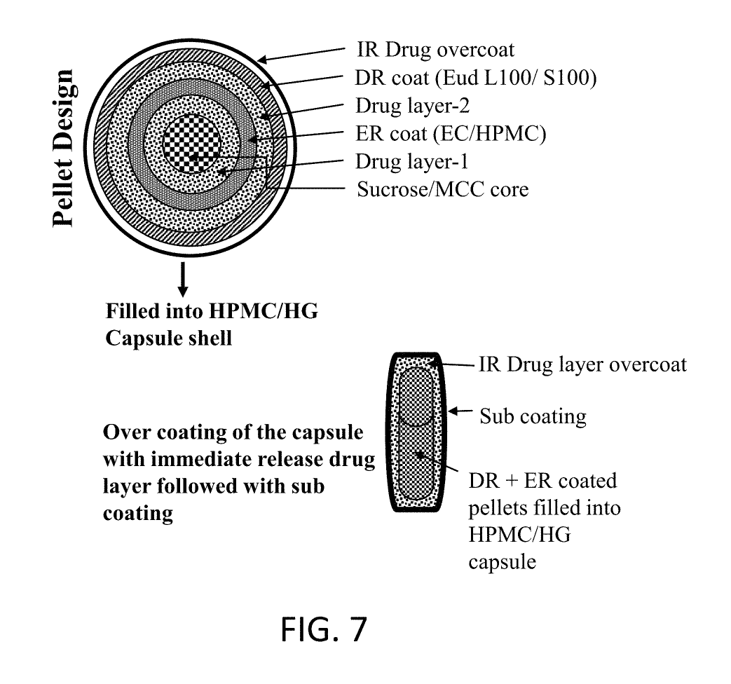

Referring to FIG. 7 and Table 13, each of the exemplary capsules comprises pulse release pellets filled in HPMC or hard gelatin capsule (similar to those shown in FIG. 5 and Table 11), and an IR overcoat comprising methylergonovine maleate, which is coated onto the HPMC or hard gelatin capsule. The pulse release pellets have the same structure as that illustrated in FIG. 5, and each comprises at least five layers including a first drug layer (“Drug layer-1”), an ER coat, a second drug layer (“Drug layer-2”), a DR coat, and an IR overcoat. The formulations for each layer are shown in Table 13. Such a pulse release capsule contains methylergonovine maleate in the range of from about 0.4 mg to about 0.7 mg in total, for example, about 0.4 mg, about 0.45 mg, about 0.6 mg, and about 0.7 mg. The capsules containing about 0.4 mg or about 0.45 mg of methylergonovine maleate are suitable for twice daily administration, and the capsules containing about 0.6 mg or about 0.7 mg of methylergonovine maleate are suitable for once daily administration.

The pulse release pellets as shown in FIG. 7 and Table 13 are manufactured following the procedures, steps 3.1-3.5, as described above. The resulting capsules are made following steps 3.6 and 3.7:

Step 3.6. Capsule filling: The pulse release pellets are filled into capsules comprising HPMC or hard gelatin. Stearic acid is used as a lubricant.

Step 3.7. Capsule coating with an IR overcoat: According to the formulation for the IR overcoat in Table 12, methylergonovine maleate, povidone, and tartaric acid are dissolved in purified water to provide a fourth dispersion solution, which is coated onto the capsules comprising HPMC or hard gelatin to provide the IR overcoat on the capsules. The resulting structure is illustrated in FIG. 7. The IR overcoat on the capsules may be also formed before the step of filling pulse release pellets into capsules comprising HPMC or hard gelatin.

TABLE 11
Pulsed Release Pellets Filled in HPMC or Hard Gelatin Capsule

| | | for twice daily | | for once daily | | % w/w of | |
|---|---|---|---|---|---|---|---|
| Sr. No. | Ingredients | 0.4 mg/Tab | 0.45 mg/Tab | 0.6 mg/Tab | 0.7 mg/Tab | total weight | Range |

Drug layer-1 (0.15 mg to 0.3 mg)

| | | | | | | | |
|---|---|---|---|---|---|---|---|
| 1 | Methylergonovine maleate USP | 0.15 | 0.2 | 0.25 | 0.3 | 0.19 | 0.01% to 10% |
| 2 | Sugar spheres, NF 30/35 mesh | 50 | 50 | 50 | 50 | 63.94 | 10% to 95% |
| 3 | Povidone | 3 | 3 | 3 | 3 | 3.84 | 0.1% to 20% |
| 4 | Tartaric acid | 0.6 | 0.6 | 0.6 | 0.6 | 0.77 | 0.01% to 10% |
| 5 | Water | q.s. | q.s. | q.s. | q.s. | . . . | . . . |

| | | | | | | |
|---|---|---|---|---|---|---|
| Weight of layer (mg) | 53.75 | 53.8 | 53.85 | 53.9 | 68.73 | . . . |

ER coat (EC/HPMC)

| | | | | | | | |
|---|---|---|---|---|---|---|---|
| 6 | Ethyl cellulose (7/10 cps) | 3 | 3 | 3 | 3 | 3.84 | 0.01% to 20% |
| 7 | HPMC (3/5 cps) | 2 | 2 | 2 | 2 | 2.56 | 0.01% to 20% |
| 8 | Methylene chloride | q.s | q.s | q.s | q.s | . . . | . . . |
| 9 | Isopropyl alcohol | q.s | q.s | q.s | q.s | . . . | . . . |

| | | | | | | |
|---|---|---|---|---|---|---|
| Weight of layer (mg) | 5 | 5 | 5 | 5 | 6.39 | . . . |

Drug layer-2 (0.15 mg to 0.3 mg)

| | | | | | | | |
|---|---|---|---|---|---|---|---|
| 10 | Methylergonovine maleate USP | 0.15 | 0.15 | 0.25 | 0.3 | 0.19 | 0.01% to 10% |
| 11 | Povidone | 5 | 5 | 5 | 5 | 6.39 | 0.1% to 20% |
| 12 | Tartaric acid | 0.6 | 0.6 | 0.6 | 0.6 | 0.77 | 0.01% to 10% |
| 13 | Water | q.s. | q.s. | q.s. | q.s. | . . . | . . . |

| | | | | | | |
|---|---|---|---|---|---|---|
| Weight of layer (mg) | 5.75 | 5.75 | 5.85 | 5.9 | 7.35 | . . . |

DR coat (Eud L100/S100)

| | | | | | | | |
|---|---|---|---|---|---|---|---|
| 14 | Eudragit L100 | 1.25 | 1.25 | 1.25 | 1.25 | 1.60 | 0.1% to 20% |
| 15 | Eudragit S100 | 3.75 | 3.75 | 3.75 | 3.75 | 4.80 | 0.1% to 20% |
| 16 | Dibutyl sebacate | 1 | 1 | 1 | 1 | 1.28 | 0.1% to 20% |
| 17 | Acetone | q.s | q.s | q.s | q.s | . . . | . . . |
| 18 | Isopropyl alcohol | q.s | q.s | q.s | q.s | . . . | . . . |
| 19 | Water | q.s | q.s | q.s | q.s | . . . | . . . |

| | | | | | | |
|---|---|---|---|---|---|---|
| Weight of layer (mg) | 6 | 6 | 6 | 6 | 7.67 | . . . |

IR drug overcoat (0.1 mg)

| 20 | Methylergonovine maleate USP | 0.1 | 0.1 | 0.1 | 0.1 | 0.13 | 0.01% to 10% |
|---|---|---|---|---|---|---|---|
| 21 | Povidone | 5 | 5 | 5 | 5 | 6.39 | 0.1% to 20% |
| 22 | Tartaric acid | 0.6 | 0.6 | 0.6 | 0.6 | 0.77 | 0.01% to 10% |
| 23 | Water | q.s. | q.s. | q.s. | q.s. | . . . | . . . |
| Weight of layer (mg) | | 5.7 | 5.7 | 5.7 | 5.7 | 7.29 | . . . |
| 24 | Stearic Acid | 2.0 | 3.0 | 4.0 | 2.0 | 2.56 | 0.1% to 20% |

| | | | | | | |
|---|---|---|---|---|---|---|
| Total weight of pellets in HPMC or Hard | 78.2 | 80.3 | 80.4 | 78.5 | 100.00 | . . . |
| Gelatine Capsule (mg) | | | | | | |

TABLE 12
Pulsed Release Pellets Compressed into Tablet

| | | for twice daily | | for once daily | | % w/w of | |
|---|---|---|---|---|---|---|---|
| Sr. No. | Ingredients | 0.4 mg/Tab | 0.45 mg/Tab | 0.6 mg/Tab | 0.7 mg/Tab | total weight | Range |

Drug layer-1

| | | | | | | | |
|---|---|---|---|---|---|---|---|
| 1 | Methylergonovine maleate USP | 0.15 | 0.2 | 0.25 | 0.3 | 0.05 | 0.01% to 10% |
| 2 | Sugar spheres, NF 30/35 mesh | 50 | 50 | 50 | 50 | 17.78 | 10% to 95% |
| 3 | Povidone | 3 | 3 | 3 | 3 | 1.07 | 0.1% to 20% |
| 4 | Tartaric acid | 0.6 | 0.6 | 0.6 | 0.6 | 0.21 | 0.01% to 10% |
| 5 | Water | q.s. | q.s. | q.s. | q.s. | . . . | . . . |

| | | | | | | |
|---|---|---|---|---|---|---|
| Weight of layer (mg) | 53.75 | 53.8 | 53.85 | 53.9 | 19.11 | . . . |

ER coat (EC/HPMC)

| | | | | | | | |
|---|---|---|---|---|---|---|---|
| 6 | Ethyl cellulose (7/10 cps) | 3 | 3 | 3 | 3 | 1.07 | 0.01% to 20% |
| 7 | HPMC (3/5 cps) | 2 | 2 | 2 | 2 | 0.71 | 0.01% to 20% |
| 8 | Methylene chloride | q.s | q.s | q.s | q.s | . . . | . . . |
| 9 | Isopropyl alcohol | q.s | q.s | q.s | q.s | . . . | . . . |

| | | | | | | |
|---|---|---|---|---|---|---|
| Weight of layer (mg) | 5 | 5 | 5 | 5 | 1.78 | . . . |

Drug layer-2

| | | | | | | | |
|---|---|---|---|---|---|---|---|
| 10 | Melhylergonovine maleate USP | 0.15 | 0.15 | 0.25 | 0.3 | 0.05 | 0.01% to 10% |
| 11 | Povidone | 5 | 5 | 5 | 5 | 1.78 | 0.1% to 20% |
| 12 | Tartaric acid | 0.6 | 0.6 | 0.6 | 0.6 | 0.21 | 0.01% to 10% |
| 13 | Water | q.s. | q.s. | q.s. | q.s. | . . . | . . . |

| | | | | | | |
|---|---|---|---|---|---|---|
| Weight of layer (mg) | 5.75 | 5.75 | 5.85 | 5.9 | 2.04 | . . . |

DR coat (Eud L100/S100)

| | | | | | | | |
|---|---|---|---|---|---|---|---|
| 14 | Eudragit L100 | 1.25 | 1.25 | 1.25 | 1.25 | 0.44 | 0.1% to 20% |
| 15 | Eudragit S100 | 3.75 | 3.75 | 3.75 | 3.75 | 1.33 | 0.1% to 20% |
| 16 | Dibutyl sebacate | 1 | 1 | 1 | 1 | 0.36 | 0.1% to 20% |
| 17 | Acetone | q.s | q.s | q.s | q.s | . . . | . . . |
| 18 | Isopropyl alcohol | q.s | q.s | q.s | q.s | . . . | . . . |
| 19 | Water | q.s | q.s | q.s | q.s | . . . | . . . |

| | | | | | | |
|---|---|---|---|---|---|---|
| Weight of layer (mg) | 6 | 6 | 6 | 6 | 2.13 | . . . |

IR drug overcoat (0.1 mg)

| 20 | Methylergonovine maleate USP | 0.1 | 0.1 | 0.1 | 0.1 | 0.04 | 0.01% to 10% |
|---|---|---|---|---|---|---|---|
| 21 | Povidone | 5 | 5 | 5 | 5 | 1.78 | 0.1% to 20% |
| 22 | Tartaric acid | 0.6 | 0.6 | 0.6 | 0.6 | 0.21 | 0.01% to 10% |
| 23 | Water | q.s. | q.s. | q.s. | q.s. | . . . | . . . |
| Weight of layer (mg) | | 5.7 | 5.7 | 5.7 | 5.7 | 2.03 | . . . |
| 24 | Total Coated Pellets | 76.2 | 76.25 | 76.4 | 76.5 | 27.10 | 0.1% to 20% |
| 25 | Microcrystalline Cellulose NF | 200 | 200 | 200 | 200 | 71.12 | 10% to 95% |
| 26 | Stearic Acid | 5.0 | 5.0 | 5.0 | 5.0 | 1.78 | 0.1% to 20% |

| | | | | | | |
|---|---|---|---|---|---|---|
| Total Tablet Weight | 281.2 | 281.3 | 281.4 | 281.5 | 100.00 | . . . |

TABLE 13
Pulsed Release Pellets Filled in HPMC or Hard Gelatin Capsule with IR Overcoat on Capsule

| | | for twice daily | | for once daily | | % w/w of | |
|---|---|---|---|---|---|---|---|
| Sr. No. | Ingredients | 0.4 mg/Tab | 0.45 mg/Tab | 0.6 mg/Tab | 0.7 mg/Tab | total weight | Range |

Drug layer-1 (0.15 mg to 0.3 mg)

| | | | | | | | |
|---|---|---|---|---|---|---|---|
| 1 | Methylergonovine maleate USP | 0.1 | 0.15 | 0.2 | 0.3 | 0.36 | 0.01% to 10% |
| 2 | Sugar spheres, NF 30/35 mesh | 50 | 50 | 50 | 50 | 59.45 | 10% to 95% |
| 3 | Povidone | 3 | 3 | 3 | 3 | 3.57 | 0.1% to 20% |
| 4 | Tartaric acid | 0.6 | 0.6 | 0.6 | 0.6 | 0.71 | 0.01% to 10% |
| 5 | Water | q.s. | q.s. | q.s. | q.s. | . . . | . . . |

| | | | | | | |
|---|---|---|---|---|---|---|
| Weight of layer (mg) | 53.7 | 53.75 | 53.8 | 53.9 | 64.09 | . . . |

ER coat (EC/HPMC)

| | | | | | | | |
|---|---|---|---|---|---|---|---|
| 6 | Ethyl cellulose (7/10 cps) | 3 | 3 | 3 | 3 | 3.57 | 0.01% to 20% |
| 7 | HPMC (3/5 cps) | 2 | 2 | 2 | 2 | 2.38 | 0.01% to 20% |
| 8 | Methylene chloride | q.s | q.s | q.s | q.s | . . . | . . . |
| 9 | Isopropyl alcohol | q.s | q.s | q.s | q.s | . . . | . . . |

| | | | | | | |
|---|---|---|---|---|---|---|
| Weight of layer (mg) | 5 | 5 | 5 | 5 | 5.95 | . . . |

Drug layer-2 (0.1 mg to 0.2 mg)

| | | | | | | | |
|---|---|---|---|---|---|---|---|
| 10 | Methylergonovine maleate USP | 0.1 | 0.1 | 0.2 | 0.2 | 0.24 | 0.01% to 10% |
| 11 | Povidone | 5 | 5 | 5 | 5 | 5.95 | 0.1% to 20% |
| 12 | Tartaric acid | 0.6 | 0.6 | 0.6 | 0.6 | 0.71 | 0.01% to 10% |
| 13 | Water | q.s. | q.s. | q.s. | q.s. | . . . | . . . |

| | | | | | | |
|---|---|---|---|---|---|---|
| Weight of layer (mg) | 5.7 | 5.7 | 5.8 | 5.8 | 6.90 | . . . |

DR coat (Eud L100/S100)

| | | | | | | | |
|---|---|---|---|---|---|---|---|
| 14 | Eudragit L100 | 1.25 | 1.25 | 1.25 | 1.25 | 1.49 | 0.1% to 20% |
| 15 | Eudragit S100 | 3.75 | 3.75 | 3.75 | 3.75 | 4.46 | 0.1% to 20% |
| 16 | Dibutyl sebacate | 1 | 1 | 1 | 1 | 1.19 | 0.1% to 20% |
| 17 | Acetone | q.s | q.s | q.s | q.s | . . . | . . . |
| 18 | Isopropyl alcohol | q.s | q.s | q.s | q.s | . . . | . . . |
| 19 | Water | q.s | q.s | q.s | q.s | . . . | . . . |

| | | | | | | |
|---|---|---|---|---|---|---|
| Weight of layer (mg) | 6 | 6 | 6 | 6 | 7.13 | . . . |

IR drug overcoat (0.1 mg)

| 20 | Methylergonovine maleate USP | 0.1 | 0.1 | 0.1 | 0.1 | 0.12 | 0.01% to 10% |
|---|---|---|---|---|---|---|---|
| 21 | Povidone | 5 | 5 | 5 | 5 | 5.95 | 0.1% to 20% |
| 22 | Tartaric acid | 0.6 | 0.6 | 0.6 | 0.6 | 0.71 | 0.01% to 10% |
| 23 | Water | q.s. | q.s. | q.s. | q.s. | . . . | . . . |
| Weight of layer (mg) | | 5.7 | 5.7 | 5.7 | 5.7 | 6.78 | . . . |
| 24 | Stearic Acid | 2.0 | 3.0 | 4.0 | 2.0 | 2.38 | 0.1% to 20% |

| | | | | | | |
|---|---|---|---|---|---|---|
| Total weight of pellets in HPMC or Hard | 78.1 | 80.2 | 82.3 | 78.4 | 93.22 | . . . |
| Gelatine Capsule (mg) | | | | | | |

IR drug layer overcoat on capsule (0.1 mg)

| | | | | | | | |
|---|---|---|---|---|---|---|---|
| 25 | Methylergonovine maleate USP | 0.1 | 0.1 | 0.1 | 0.1 | 0.12 | 0.01% to 10% |
| 26 | Povidone | 5 | 5 | 5 | 5 | 5.95 | 0.1% to 20% |
| 27 | Tartaric acid | 0.6 | 0.6 | 0.6 | 0.6 | 0.71 | 0.01% to 10% |
| 28 | Water | q.s. | q.s. | q.s. | q.s. | . . . | . . . |

| | | | | | | |
|---|---|---|---|---|---|---|
| Weight of layer (mg) | 5.7 | 5.7 | 5.7 | 5.7 | 6.78 | . . . |
| Total weight of pellets and over coat on HPMC | 84 | 85 | 86 | 84 | 100.00 | . . . |
| or Hard Gelatin Capsule (mg) | | | | | | |

### Example 13

Tablets Comprising IR Core and ER or Osmotic Coat

In accordance with some embodiments, the present disclosure provides an oral modified release tablet comprising an IR core (as a reservoir) having methylergonovine or a pharmaceutically acceptable salt and an ER coat or osmotic coat. Such a tablet may further comprise an IR overcoat containing methylergonovine. The resulting tablet comprises methylergonovine or a pharmaceutically acceptable salt thereof in the range of from about 0.4 mg to about 0.7 mg in total, which is suitable for twice daily or once daily oral administration.

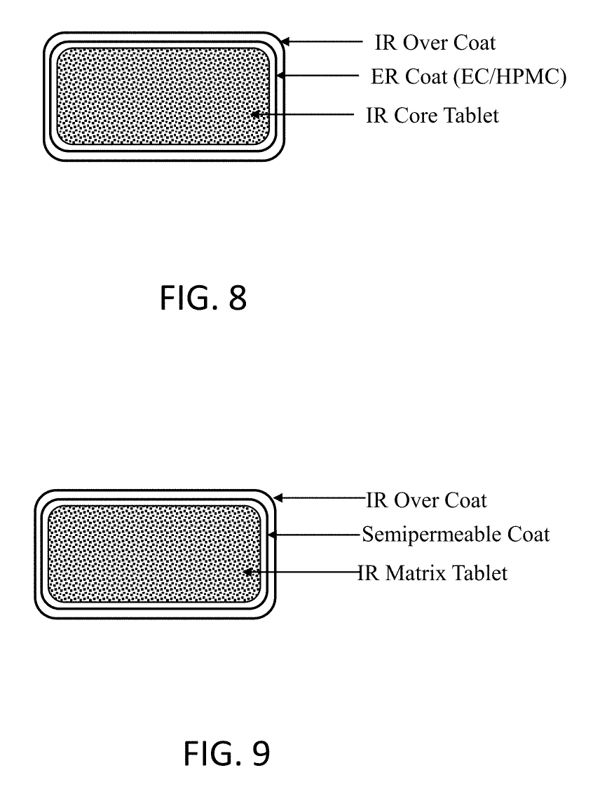

Tables 14 and 15 show two exemplary types of such tablets having structure as shown in FIGS. 8-9, respectively.

Referring to FIG. 8 and Table 14, each of the exemplary tablets comprises an immediate release (“IR”) core (or called a core tablet or a reservoir), an extended release (“ER”) coat, and an immediate release (“IR”) overcoat. The formulations for each layer are shown in Table 14. Each tablet in Table 14 contains methylergonovine maleate in the range of from about 0.4 mg to about 0.7 mg in total, for example, 0.4 mg, 0.45 mg, 0.6 mg, and 0.7 mg. The tablets containing about 0.4 mg or about 0.45 mg of methylergonovine maleate are suitable for twice daily (“BID”) administration, and the tablets containing about 0.6 mg or about 0.7 mg of methylergonovine maleate are suitable for once daily (“OD”) administration.

The tablets in Table 14 are manufactured through the steps as follows:

Step 4.1. Preparation of core tablets: Lactose monohydrate, microcrystalline cellulose, and corn starch are co-sifted through a suitable sieve to provide a blend. Separately, a solution of methylergonovine maleate, povidone, and tartaric acid is prepared in purified water to provide a granulating fluid. The blend is mixed in a rapid mixer granulator (“RMG”), and then granulated with the granulating fluid. The blend is dried and passed through a co-mill using a suitable screen. These granules are mixed with stearic acid in a blender, and compressed into tablets to provide the core tablets.

Step 4.2. Coating with ER coat: According to the formulation for the ER coat in Table 14, ethyl cellulose and HPMC are dissolved in a mixture of methylene chloride and isopropyl alcohol to provide a solution of the ER coat. The core tablets are coated with the solution for the ER coat to obtain required a weight gain.

Step 4.3. Coating of the IR overcoat: According to the formulation for the IR overcoat in Table 14, methylergonovine maleate, povidone, and tartaric acid are dissolved in purified water to provide a solution. Additional povidone was then added into the solution with continuous stirring. The solution is coated onto e tablets from step 4.3. The resulting tablets have a structure as illustrated in FIG. 8.

Referring to FIG. 9 and Table 15, each of the exemplary tablets comprises an IR core (or called a core tablet or a reservoir), an osmotic coat, and an IR overcoat. The osmotic coat is a semipermeable polymer membrane. The formulations for each layer are shown in Table 15. Each tablet in Table 15 contains methylergonovine maleate in the range of from about 0.4 mg to about 0.7 mg in total, for example, 0.4 mg, 0.45 mg, 0.6 mg, and 0.7 mg. The tablets containing 0.4 mg or 0.45 mg of methylergonovine maleate are suitable for twice daily administration, and the tablets containing 0.6 mg or 0.7 mg of methylergonovine maleate are suitable for once daily administration.

The tablets in Table 15 are manufactured through the steps as follows:

Step 5.1. Preparation of core tablets: Lactose monohydrate, microcrystalline cellulose, mannitol, polyethylene oxide, and corn starch are co-sifted through a suitable sieve to provide a blend. Separately, a solution of methylergonovine maleate, povidone, and tartaric acid is prepared in purified water to provide a granulating fluid. The blend is mixed in a rapid mixer granulator (“RMG”), and then granulated with the granulating fluid. The blend is dried and passed through a co-mill using a suitable screen. These granules are mixed with stearic acid in a blender, and compressed into tablets to provide the core tablets.

Step 5.2. Coating with osmotic coat: According to the formulation for the semipermeable coat (osmotic coat) in Table 15, cellulose acetate phthalate and optionally povidone are dissolved in a mixture of acetone and water to provide a solution of the osmotic coat. The core tablets are coated with the solution for the osmotic coat to obtain required a weight gain under suitable coating conditions.

Step 5.3. Laser drilling (an optional step): the tablets from step 5.2 are drilled by laser to form orifice(s) for allowing the release of drug from the core.

Step 5.4. Coating of the IR overcoat: According to the formulation for the IR overcoat in Table 15, methylergonovine maleate, and tartaric acid are dissolved in purified water to provide a solution. Povidone was then added into the solution with continuous stirring. The solution is coated onto the tablets from step 5.2 or 5.3. The resulting tablets have a structure as illustrated in FIG. 9.

TABLE 14
IR Reservoir with ER coal Tablet with IR Overcoat

| | | for twice daily | | for once daily | | % w/w of | |
|---|---|---|---|---|---|---|---|
| Sr. No. | Ingredients | 0.4 mg/Tab | 0.45 mg/Tab | 0.6 mg/Tab | 0.7 mg/Tab | total weight | Range |

IR Core Tablet

| | | | | | | | |
|---|---|---|---|---|---|---|---|
| 1 | Methylergonovine maleate USP | 0.3 | 0.35 | 0.2 | 0.5 | 0.27 | 0.01% to 10% |
| 2 | Microcrystalline cellulose | 35.1 | 35.05 | 35.2 | 34.9 | 31.14 | 2% to 75% |
| 3 | Lactose monohydrate | 52 | 52 | 52 | 52 | 46.14 | 2% to 75% |
| 4 | Povidone | 3 | 3 | 3 | 3 | 2.66 | 0.1% to 20% |
| 5 | Corn starch | 5 | 5 | 5 | 5 | 4.44 | 0.1% to 20% |
| 6 | Tartaric acid | 0.6 | 0.6 | 0.6 | 0.6 | 0.53 | 0.01% to 10% |
| 7 | Stearic acid | 4 | 4 | 4 | 4 | 3.55 | 0.1% to 20% |
| 8 | Water | q.s. | q.s. | q.s. | q.s. | . . . | . . . |

| | | | | | | |
|---|---|---|---|---|---|---|
| Weight of core tablet (mg) | 100 | 100 | 100 | 100 | 88.73 | . . . |

ER coat

| | | | | | | | |
|---|---|---|---|---|---|---|---|
| 9 | Ethyl cellulose (7/10 cps) | 5 | 5 | 5 | 5 | 4.44 | 0.1% to 20% |
| 10 | HPMC (3/5 cps) | 2 | 2 | 2 | 2 | 1.77 | 0.1% to 20% |
| 11 | Methylene chloride | q.s | q.s | q.s | q.s | . . . | . . . |
| 12 | Isopropyl alcohol | q.s | q.s | q.s | q.s | . . . | . . . |

| | | | | | | |
|---|---|---|---|---|---|---|
| Weight of ER coat (mg) | 7 | 7 | 7 | 7 | 6.21 | . . . |

IR Overcoat

| | | | | | | | |
|---|---|---|---|---|---|---|---|
| 13 | Methylergonovine maleate USP | 0.1 | 0.1 | 0.1 | 0.1 | 0.09 | 0.01% to 10% |
| 14 | Povidone | 5 | 5 | 5 | 5 | 4.44 | 0.1% to 20% |
| 15 | Tartaric acid | 0.6 | 0.6 | 0.6 | 0.6 | 0.53 | 0.1% to 20% |
| 16 | Water | q.s | q.s | q.s | q.s | . . . | . . . |

| | | | | | | |
|---|---|---|---|---|---|---|
| Weight of IR overcoat (mg) | 5.7 | 5.7 | 5.7 | 5.7 | 5.06 | . . . |
| Total weight of Tablet (mg) | 112.7 | 112.7 | 112.7 | 112.7 | 100.00 | . . . |

TABLE 15
Osmotic Tablet with IR Overcoat

| | | for twice daily | | for once daily | | % w/w of | |
|---|---|---|---|---|---|---|---|
| Sr. No. | Ingredients | 0.4 mg/Tab | 0.45 mg/Tab | 0.6 mg/Tab | 0.7 mg/Tab | total weight | Range |

IR Core Tablet (0.3 mg to 0.6 mg)

| | | | | | | | |
|---|---|---|---|---|---|---|---|
| 1 | Methylergonovine maleate USP | 0.3 | 0.35 | 0.5 | 0.6 | 0.20 | 0.01% to 10% |
| 2 | Microcrystalline cellulose | 25 | 25 | 25 | 25 | 16.81 | 2% to 75% |
| 3 | Lactose monohydrate | 32.1 | 32.05 | 31.9 | 31.8 | 21.59 | 2% to 75% |
| 4 | Povidone | 3 | 3 | 3 | 3 | 2.02 | 0.1% to 20% |
| 5 | Polyethylene Oxide | 25 | 25 | 25 | 25 | 16.81 | 1% to 50% |
| 6 | Mannitol | 30 | 30 | 30 | 30 | 20.17 | 1% to 50% |
| 7 | Corn starch | 5 | 5 | 5 | 5 | 3.36 | 0.1% to 20% |
| 8 | Tartaric acid | 0.6 | 0.6 | 0.6 | 0.6 | 0.40 | 0.1% to 20% |
| 9 | Stearic acid | 4 | 4 | 4 | 4 | 2.69 | 0.1% to 20% |
| 10 | Water | q.s. | q.s. | q.s. | q.s. | . . . | . . . |

| | | | | | | |
|---|---|---|---|---|---|---|
| Weight of core tablet (mg) | 125 | 125 | 125 | 125 | 84.06 | . . . |

Semipermeable coat

| | | | | | | | |
|---|---|---|---|---|---|---|---|
| 11 | Cellulose acetate phthalate | 15 | 15 | 15 | 15 | 10.09 | 1% to 50% |
| 12 | Povidone | 3 | 3 | 3 | 3 | 2.02 | 0.1% to 20% |
| 13 | Acetone | q.s | q.s | q.s | q.s | . . . | . . . |
| 14 | Water | q.s | q.s | q.s | q.s | . . . | . . . |

| | | | | | |
|---|---|---|---|---|---|
| Weight of ER coat (mg) | 18 | 18 | 18 | 18 | 12.10 |

IR Overcoat (0.1 mg)

| | | | | | | | |
|---|---|---|---|---|---|---|---|
| 15 | Methylergonovine maleate USP | 0.1 | 0.1 | 0.1 | 0.1 | 0.07 | 0.01% to 10% |
| 16 | Povidone | 5 | 5 | 5 | 5 | 3.36 | 0.1% to 20% |
| 17 | Tartaric acid | 0.6 | 0.6 | 0.6 | 0.6 | 0.40 | 0.1% to 20% |
| 18 | Water | q.s | q.s | q.s | q.s | . . . | . . . |

| | | | | | | |
|---|---|---|---|---|---|---|
| Weight of IR overcoat (mg) | 5.7 | 5.7 | 5.7 | 5.7 | 3.83 | . . . |
| Total weight of Tablet (mg) | 148.7 | 148.7 | 148.7 | 148.7 | 100.00 | . . . |

Dissolution Specification and Testing:

The dissolution study was carried out using a USP type II dissolution apparatus (with paddle stirring element) at 75 rpm, 37° C., in 900 mL of dissolution medium containing tartaric acid (1 in 200), as per the current version of USP monograph, Dissolution <711>, using the USP Reference standard <11>-USP methylergonovine maleate RS. After a time interval (e.g., 0.5, 1, 2, 3, 6, 10, 16 hrs), the dissolution of methylergonovine maleate is tested. A portion of the solution under test is filtered into a flask. The intensity of this solution is concomitantly determined, in comparison with a standard solution of USP methylergonovine maleate RS in the same medium having a known concentration that of sample concentration. The intensity is tested by stability indicating HPLC method with a wavelength of 315 nm, using tartaric acid solution (1 in 200) as the blank.

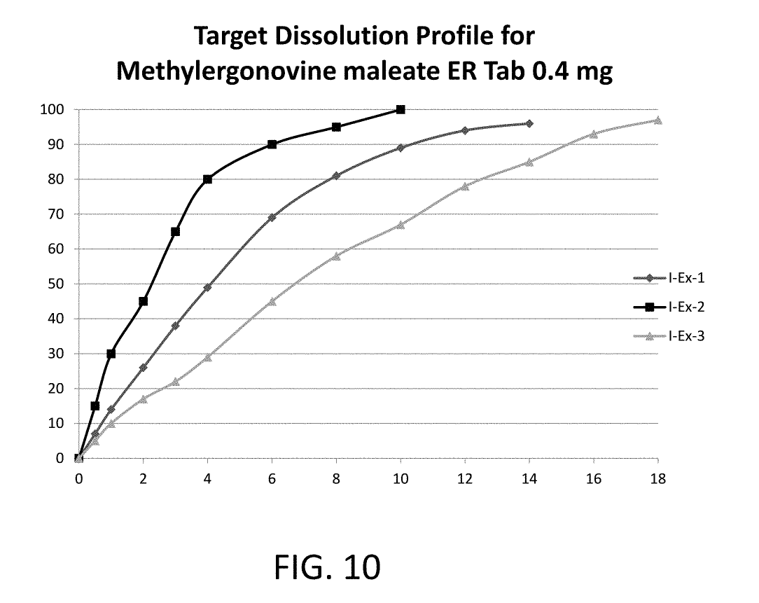

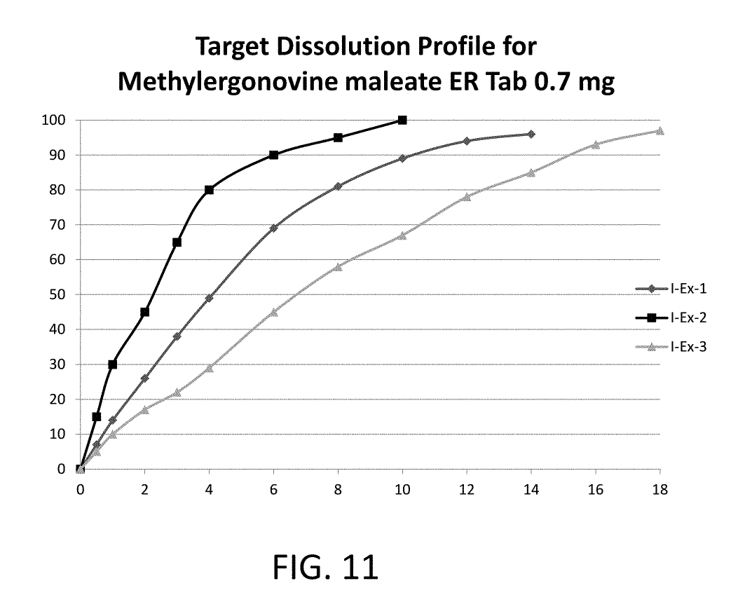

The formulations and resulting tablet or capsules are evaluated in comparison with a dissolution specification for once daily or twice daily administration as shown in Table 16. FIG. 10 shows the dissolution profiles shown by three examples (I-Ex-1, I-Ex-2, and I-Ex-3) of the exemplary monolithic matrix tablet (as illustrated in FIG. 1) comprising 0.4 mg of methylergonovine. FIG. 11 shows the dissolution profiles shown by three examples (I-Ex-1, I-Ex-2, and I-Ex-3) of the exemplary monolithic matrix tablet (as illustrated in FIG. 1) comprising 0.7 mg of methylergonovine.

The present disclosure also provides the method of making the formulations and the dosage forms as described herein, and the method of using the formulations and the dosage forms. The formulations and dosage forms described herein can be used for treating a methylergonovine responsive condition selected from migraine, refractory migraine, uterine atony, uterine haemorrhage, subinvolution of the uterus, or uterine haemorrhage in the second stage of labor. Depending on the dosage amount, the dosage forms can be administrated once or twice daily orally.

TABLE 16
Target Dissolution Specification
Dissolution Condition: Tartaric acid solution (1 in 200);
900 mL. USP-2 (Paddle), 75 rpm

Dissolution Specification

| Time | For BID (0.4 mg dose) | For OD (0.7 mg dose) |
|---|---|---|
| 30 min | between 10-35% | between 5-25% |
| 2 hr | . . . | between 15-45% |
| 3 hr | between 30-60% | . . . |
| 6 hr | between 40-85% | between 25-65% |
| 10 hr | Not Less Than 70% | between 35-85% |
| 16 hr | . . . | Not Less Than 75% |

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A solid pharmaceutical oral composition configured for once or twice daily administration, comprising pulsed release pellets, each pellet comprising at least
a first drug layer comprising methylergonovine or a pharmaceutically acceptable salt thereof; and
a second drug layer comprising methylergonovine or a pharmaceutically acceptable salt thereof,
wherein the solid pharmaceutical oral composition comprises from about 0.3 mg to about 0.8 mg in total of methylergonovine or a pharmaceutically acceptable salt thereof.

2. The solid pharmaceutical oral composition of claim 1, wherein each pellet further comprises a sugar core, the first drug layer disposed on and covering the sugar core.

3. The solid pharmaceutical oral composition of claim 1, wherein each pellet further comprises:
an extended release coating comprising at least a first release rate controlling polymer disposed between the first drug layer and the second drug layer; and
a delayed release coating comprising at least a second release rate controlling polymer disposed on the second drug layer.

4. The solid pharmaceutical oral composition of claim 3, wherein the first release rate controlling polymer comprises one or more hydrophilic release rate controlling compound, one or more hydrophobic release rate controlling compound, or combinations thereof.

5. The solid pharmaceutical oral composition of claim 4, wherein the first release rate controlling polymer in the extended release coating comprises hydroxypropyl methylcellulose and ethyl cellulose.

6. The solid pharmaceutical oral composition of claim 3, wherein the second release rate controlling polymer in the delayed release coating comprises at least one copolymer of methacrylic acid and methyl methacrylate.

7. The solid pharmaceutical oral composition of claim 3, wherein each pellet further comprises an immediate release layer comprising methylergonovine or a pharmaceutically acceptable salt thereof disposed on and covering the delayed release coating.

8. The solid pharmaceutical oral composition of claim 1, wherein the solid pharmaceutical oral composition is in a form of a tablet, the tablet comprising a plurality of compressed pulsed release pellets.

9. The solid pharmaceutical oral composition of claim 1, wherein the solid pharmaceutical oral composition is a in a form of a capsule filled with a plurality of pulsed release pellets.

10. The solid pharmaceutical oral composition of claim 1, wherein the solid pharmaceutical oral composition is a capsule filled with a plurality of pulsed release pellets, and the capsule comprises an exterior immediate release coating comprising methylergonovine or a pharmaceutically acceptable salt thereof.

11. The solid pharmaceutical oral composition of claim 1, wherein the pharmaceutically acceptable salt of methylergonovine is maleate.

12. The solid pharmaceutical oral composition of claim 1, wherein the solid pharmaceutical oral composition comprises from about 0.6 mg to about 0.7 mg in total of methylergonovine or a pharmaceutically acceptable salt thereof, and is configured for once daily administration.

13. The solid pharmaceutical oral composition of claim 12, wherein the solid pharmaceutical oral composition is configured to provide a dissolution profile of a release of methylergonovine or a pharmaceutically acceptable salt thereof
within 0.5 hours being between about 5% and about 25%,
within 2 hours being between about 15% and about 45%,
within 6 hours being between about 25% and about 65%,
within 10 hours being between about 35% and about 85%, and
within 16 hours being not less than 75%,
as measured by a dissolution method employing a USP Type-II dissolution apparatus equipped with a paddle, a rotation speed of 75 rpm and 900 mL of tartaric acid (1 in 200 w/w) as dissolution medium.

14. The solid pharmaceutical oral composition of claim 1, wherein the solid pharmaceutical oral composition comprises from about 0.4 mg to about 0.45 mg in total of methylergonovine or a pharmaceutically acceptable salt thereof, and is configured for twice daily administration.

15. The solid pharmaceutical oral composition of claim 14, wherein the solid pharmaceutical oral composition is configured to provide a dissolution profile of a release of methylergonovine or a pharmaceutically acceptable salt thereof
within 0.5 hours being between about 10% and about 35%,
within 3 hours being between about 30% and about 60%,
within 6 hours being between about 40% and about 85%, and
within 10 hours being not less than 70%,
as measured by a dissolution method employing a USP Type-II dissolution apparatus equipped with a paddle, a rotation speed of 75 rpm and 900 mL of tartaric acid (1 in 200 w/w) as dissolution medium.

16. A method of making the solid pharmaceutical oral composition of claim 1, comprising preparing pulsed release pellets comprising methylergonovine or a pharmaceutically acceptable salt thereof.

17. The method of claim 16, further comprising compressing the pulsed release pellets into a tablet.

18. The method of claim 16, further comprising filling the pulsed release pellets into a capsule.

19. The method of claim 16, further comprising filling the pulsed release pellets into a capsule, and coating the capsule with an exterior immediate release coating comprising methylergonovine or a pharmaceutically acceptable salt thereof.

20. A method for treating a subject having a methylergonovine responsive condition comprising a step of administering to the subject in need thereof a therapeutic effective amount of the solid pharmaceutical oral composition of claim 1.

21. The method for treating a subject having a methylergonovine responsive condition of claim 20, wherein the methylergonovine responsive condition is selected from migraine, refractory migraine, uterine atony, uterine haemorrhage, subinvolution of the uterus, or uterine haemorrhage in the second stage of labor.
